(19) United States
(12) Patent Application Publication
(10) Pub. No.: US 2025/0179442 A1
(43) Pub. Date: Jun. 5, 2025

(54) METHOD OF PRODUCING ADENO-ASSOCIATED VIRUS

(71) Applicant: KANEKA CORPORATION, Osaka (JP)

(72) Inventors: Hisako YAURA, Hyogo (JP); Kazunobu MINAKUCHI, Hyogo (JP); Masakatsu NISHIHACHIJO, Hyogo (JP); Masahiro ARATAKE, Hyogo (JP); Takuma SUEOKA, Hyogo (JP)

(21) Appl. No.: 18/841,498

(22) PCT Filed: Mar. 16, 2023
(86) PCT No.: PCT/JP2023/010298

§ 371 Date: Aug. 26, 2024

(30) Foreign Application Priority Data
Mar. 29, 2022 (JP) 2022-054065

(51) Int. Cl. C12N 7/00; B01D 15/38

(52) U.S. Cl. CPC C12N 7/00; B01D 15/3809; C12N 2750/14151

(57) ABSTRACT

A method of producing an adeno-associated virus (AAV) includes separating an adeno-associated virus (AAV), which is bonded to an affinity carrier, from the affinity carrier using an amino acid-containing solution of pH 3 or higher and PH 7 or lower.

### TECHNICAL FIELD

The present invention relates to a method of producing an adeno-associated virus.

### BACKGROUND ART

Adeno-associated virus (AAV) vectors are important vectors in the field of gene therapy. AAV vectors are produced through culturing of animal cells or insect cells. Upon use of AAV vectors, there is a need for a method of purifying AAV vectors from a large amount of impurities derived from cells, while retaining biological activity.

For purification of AAV vectors, purification using an affinity chromatography carrier is generally known, and the affinity chromatography carrier can specifically adsorb and purify AAV vectors from a cell culture solution (Patent Document 1).

However, the purification method of the related art needs to use a low pH (lower than pH 3, such as pH 2.6) solution when an AAV vector is eluted from a carrier at a high yield (Non-Patent Document 1). However, it has been known that the biological activity (titer) of the AAV vector is lowered in the low pH (lower than pH 3, such as pH 2.6) solution (Non-Patent Document 2), which brings a challenge in effective purification of an AAV vector while retaining the titer.

Accordingly, a method for efficiently purifying AAV while retaining the biological activity in the condition of pH 3 or higher and pH 7 or lower has not been known at all, and development of such method is strongly desired.

### CITATION LIST

### Patent Document

Patent Document 1: PCT Japanese Translation Application Publication No. 2021-508484

### Non-Patent Documents

Non-Patent Document 1: Mol Ther Methods Clin Dev. 2020 19:362-373
Non-Patent Document 2: Viruses 2020 12:668

### SUMMARY OF INVENTION

### Technical Problem

The present invention aims to solve the above various problems existing in the related art and to achieve the following object. Specifically, the present invention provides a method of purifying AAV while retaining biological activity in the condition of pH 3 or higher and pH 7 or lower.

### Solution to Problem

The present inventors diligently conducted research to achieve the above object. As a result, the present inventors have found the following insights. That is, a method of efficiently purifying AAV while retaining biological activity of AAV in the condition of pH 3 or higher and pH 7 or lower can be provided by: a method of producing an adeno-associated virus (AAV), where the method includes separating of an adeno-associated virus (AAV) bonded to an affinity carrier from the affinity carrier using an amino acid-containing solution of pH 3 or higher and pH 7 or lower; a method of purifying an adeno-associated virus (AAV), where the method includes separating of an adeno-associated virus (AAV) bonded to an affinity carrier from the affinity carrier using an amino acid-containing solution of pH 3 or higher and pH 7 or lower; a method of inhibiting a reduction of an infectious titer of an adeno-associated virus (AAV), where the method includes separating of an adeno-associated virus (AAV) bonded to an affinity carrier from the affinity carrier using an amino acid-containing solution of pH 3 or higher and pH 7 or lower; or an eluent for separating an adeno-associated virus (AAV) bonded to an affinity carrier from the affinity carrier, where the eluent includes an amino acid-containing solution of pH 3 or higher and pH 7 or lower.

The present invention is based on the above insights of the present inventors, and means for solving the above problems are as follows.

<1> A method of producing an adeno-associated virus (AAV) includes
separating an adeno-associated virus (AAV), which is bonded to an affinity carrier, from the affinity carrier using an amino acid-containing solution of pH 3 or higher and pH 7 or lower.

<2> A method of purifying an adeno-associated virus (AAV) includes
separating an adeno-associated virus (AAV), which is bonded to an affinity carrier, from the affinity carrier using an amino acid-containing solution of pH 3 or higher and pH 7 or lower.

<3> A method of inhibiting a reduction in an infectious titer of an adeno-associated virus (AAV) includes
separating an adeno-associated virus (AAV), which is bonded to an affinity carrier, from the affinity carrier using an amino acid-containing solution of pH 3 or higher and pH 7 or lower.

<4> An eluent, which is for separating an adeno-associated virus (AAV) bonded to an affinity carrier from the affinity carrier, includes
an amino acid-containing solution of pH 3 or higher and pH 7 or lower.

### Effects of Invention

According to the present invention, the various problems existing in the related art can be solved, the object can be achieved, and a method of purifying AAV while retaining biological activity in the condition of pH 3 or higher and pH 7 or lower can be provided.

### BRIEF DESCRIPTION OF THE DRAWINGS

FIG. 1 is a graph depicting recovery rates of AAV recovered from elution fractions and a wash fraction (strip fraction) using the purified liquid in Example 1.

FIG. 2 is a graph depicting recovery rates of AAV recovered from elution fractions using clarified liquids in Example 2, Comparative Example 1, and Comparative Example 2.

FIG. 3 is a graph depicting recovery rates of AAV recovered from elution fractions using clarified liquids in Example 3, Example 4, Example 5, and Comparative Example 3.

FIG. 4 is a graph depicting recovery rates of AAV recovered from elution fractions using clarified liquids in Example 6, Comparative Example 4, and Comparative Example 5.

FIG. 5 is a graph depicting recovery rates of AAV recovered from elution fractions using clarified liquids in Example 7, Comparative Example 4, and Comparative Example 6.

FIG. 6 is a graph depicting titers of AAV purified under corresponding pH conditions in Example 7 and Comparative Example 4.

FIG. 7 is a graph depicting AAV amounts recovered from elution fractions using clarified liquids in Example 8 in comparison with AAV amounts recovered from elution fractions using clarified liquids in Comparative Example 7.

FIG. 8 is a graph depicting an AAV amount recovered from an elution fraction using a clarified liquid in Example 9 in comparison with an AAV amount recovered from an elution fraction using a clarified liquid in Comparative Example 8.

FIG. 9 is a graph depicting AAV amounts recovered from elution fractions using clarified liquids in Example 10 in comparison with AAV amounts recovered from elution fractions using clarified liquids in Comparative Example 9.

FIG. 10 is a graph depicting AAV amounts recovered from elution fractions using clarified liquids in Example 11 in comparison with AAV amounts recovered from elution fractions using clarified liquids in Comparative Example 10.

FIG. 11 is a graph depicting titers of AAV purified under corresponding pH conditions in Example 12, represented by ratios to Comparative Example 11.

FIG. 12 is a graph depicting amounts of AAV recovered from elution fractions using clarified liquids in Examples 13 to 20 in comparison with an amount of AAV recovered from an elution fraction using a clarified liquid in Comparative Example 12.

### DESCRIPTION OF EMBODIMENTS

### (Method of Producing Adeno-Associated Virus (AAV))

The method of producing an adeno-associated virus (AAV) includes a separation step, and may further include other steps.

### —Separation Step—

The separation step is a step of separating an adeno-associated virus (AAV), which is bonded to an affinity carrier, from the affinity carrier using an amino acid-containing solution of pH 3 or higher and pH 7 or lower.

### ——Amino Acid-Containing Solution of pH 3 or Higher and pH 7 or Lower——

The amino acid-containing solution of pH 3 or higher and pH 7 or lower can be used as an eluent for separating an adeno-associated virus (AAV), which is bonded to an affinity carrier, from the affinity carrier.

The amino acid-containing solution of pH 3 or higher and pH 7 or lower includes an amino acid and may further include other components.

### ———Amino Acid———

Typically, amino acids are classified into basic amino acids, acidic amino acids, aliphatic amino acids, uncharged amino acids, aromatic amino acids, and cyclic amino acids.

Examples of the basic amino acids include arginine, histidine, and lysine.

Examples of the acidic amino acids include aspartic acid and glutamic acid.

Examples of the aliphatic amino acids include glycine, alanine, isoleucine, leucine, methionine, and valine.

Examples of the uncharged amino acids include asparagine, glutamine, serine, cysteine, and threonine.

Examples of the aromatic amino acids include phenylalanine, tryptophan, and tyrosine.

Examples of the cyclic amino acids include proline.

The amino acid for use in the present invention is not particularly limited, and may be appropriately selected according to the intended purpose. In view of efficient purification of AAV, the amino acid is preferably a basic amino acid, an acidic amino acid, an aliphatic amino acid, an uncharged amino acid, or an aromatic amino acid, more preferably a basic amino acid, an acidic amino acid, an aliphatic amino acid, or an aromatic amino acid, and yet more preferably an acidic amino acid, an aliphatic amino acid, or an aromatic amino acid. The above amino acids may be used alone or in combination.

The basic amino acid is not particularly limited, and may be appropriately selected according to the intended purpose. In view of efficient purification of AAV, the basic amino acid is preferably arginine or histidine.

The acidic amino acid is not particularly limited, and may be appropriately selected according to the intended purpose. In view of efficient purification of AAV, aspartic acid or glutamic acid is preferred.

The aliphatic amino acid is not particularly limited, and may be appropriately selected according to the intended purpose. In view of efficient purification of AAV, glycine, alanine, isoleucine, or leucine is preferred.

The uncharged amino acid is not particularly limited, and may be appropriately selected according to the intended purpose. In view of efficient purification of AAV, threonine is preferred.

The aromatic amino acid is not particularly limited, and may be appropriately selected according to the intended purpose. In view of efficient purification of AAV, tryptophan is preferred.

The amino acid is not particularly limited, and may be appropriately selected according to the intended purpose. The amino acid may be in a form of a free-form amino acid, or may be in a form of a hydrate or a salt. The salt is preferably a hydrochloric acid salt or a sodium salt in view of abundant availability.

A lower limit of a concentration of the amino acid is not particularly limited, and may be appropriately selected according to the intended purpose. In view of efficient purification of AAV, the lower limit of the concentration of the amino acid is preferably 0.1 mM or greater, more preferably 1 mM or greater, yet more preferably 5 mM or greater, yet further more preferably 10 mM or greater, particularly preferably 50 mM or greater, and most preferably 80 mM or greater.

An upper limit of the concentration of the amino acid is not particularly limited, and may be appropriately selected according to the intended purpose. In view of efficient purification of AAV, the upper limit of the concentration of the amino acid is preferably 10 M or less, more preferably 5 M or less, yet more preferably 1 M or less, yet further more preferably 500 mM or less, and particularly preferably 250 mM or less.

### Other Components

The above other components are not particularly limited, and may be appropriately selected according to the intended purpose. Examples of the above other components include a pH adjuster, a surfactant, an organic solvent, magnesium chloride, and the like.

The pH adjuster is not particularly limited, except that the pH adjuster is a substance other than the amino acid and can adjust pH to 3 or higher and 7 or lower. The pH adjuster may be appropriately selected according to the intended purpose. The pH adjuster is preferably an acid, an acid salt, or a base, more preferably an acid or an acid salt, and yet more preferably an acid.

The acid is not particularly limited, and may be appropriately selected according to the intended purpose. In view of typical use in biochemistry, the acid is preferably citric acid, succinic acid, acetic acid, malic acid, lactic acid, carbonic acid, ascorbic acid, tartaric acid, phytic acid, gluconic acid, fumaric acid, formic acid, propionic acid, butyric acid, valeric acid, caproic acid, maleic acid, phthalic acid, boric acid, or phosphoric acid. Among the above-listed acids, in view of use in chromatography purification, the acid is more preferably citric acid, succinic acid, acetic acid, malic acid, lactic acid, carbonic acid, ascorbic acid, tartaric acid, gluconic acid, fumaric acid, formic acid, propionic acid, maleic acid, phthalic acid, boric acid, or phosphoric acid, yet more preferably acetic acid, malic acid, or phosphoric acid, and particularly preferably acetic acid.

The acid salt is not particularly limited, and may be appropriately selected according to the intended purpose. In view of typical use in chromatography purification, the acid salt is preferably a citric acid salt, a succinic acid salt, an acetic acid salt, a carbonic acid salt, a tartaric acid salt, a fumaric acid salt, or a phosphoric acid salt, more preferably an acetic acid salt or a phosphoric acid salt, and yet more preferably an acetic acid salt.

A salt of the acid salt is not particularly limited, and may be appropriately selected according to the intended purpose. The salt is preferably a sodium salt or a potassium salt, and more preferably a sodium salt.

The base is not particularly limited, and may be appropriately selected according to the intended purpose. In view of typical use in chromatography purification, the base is preferably Good's buffers.

The Good's buffers are not particularly limited, and may be appropriately selected according to the intended purpose. Examples of the Good's buffer include HEPES (4-(2-hydroxyethyl)-1-piperazineethanesulfonic acid)-sodium hydroxide, MOPS (3-morpholinopropanesulfonic acid)-sodium hydroxide, MES (2-morpholinoethanesulfonic acid)-sodium hydroxide, and the like.

The above pH adjusters may be used alone or in combination.

Among the above-listed examples, the same type of an acid and an acid salt is preferably used in combination. For example, adjustment of pH using an acetic acid is not particularly limited, and may be appropriately selected according to the intended purpose, but the pH adjustment is preferably performed with two fluids that are an acetic acid and sodium acetate.

When the pH adjustment cannot be performed by the method of using the same type of an acid and an acid salt, the pH can be adjusted using hydrochloric acid or a hydroxide, such as sodium hydroxide or the like.

A lower limit of a concentration of the pH adjuster is not particularly limited, and may be appropriately selected according to the intended purpose. In view of efficient purification of AAV, the lower limit of the concentration of the pH adjuster is preferably 0.1 mM or greater, more preferably 1 mM or greater, yet more preferably 5 mM or greater, and particularly preferably 10 mM or greater.

An upper limit of the concentration of the pH adjuster is not particularly limited, and may be appropriately selected according to the intended purpose. In view of efficient purification of AAV, the upper limit of the concentration of the pH adjuster is preferably 10 M or less, more preferably 5 M or less, yet more preferably 1 M or less, particularly preferably 500 mM or less, and most preferably 250 mM or less.

The surfactant is not particularly limited, and may be appropriately selected according to the intended purpose. The surfactant is preferably a nonionic surfactant or an anionic surfactant.

The nonionic surfactant is not particularly limited, and may be appropriately selected according to the intended purpose. The nonionic surfactant is preferably Triton X100, Tween80, or Poloxamer, and more preferably Poloxamer.

The anionic surfactant is not particularly limited, and may be appropriately selected according to the intended purpose. The anionic surfactant is preferably sarkosyl.

A lower limit of a concentration of the surfactant is not particularly limited, and may be appropriately selected according to the intended purpose. In view of efficient purification of AAV, the lower limit of the concentration of the surfactant is preferably 0.001% or greater, more preferably 0.01% or greater, yet more preferably 0.1%, particularly preferably 0.015% or greater, and the most preferably 0.02% or greater.

An upper limit of a concentration of the surfactant is not particularly limited, and may be appropriately selected according to the intended purpose. In view of efficient purification of AAV, the upper limit of the concentration of the surfactant is preferably 10% or less, more preferably 5% or less, yet more preferably 1% or less, particularly preferably 0.5% or less, and the most preferably 0.25% or less.

The organic solvent is not particularly limited, and may be appropriately selected according to the intended purpose. The organic solvent is preferably ethylene glycol, DMSO, sucrose, trehalose, sorbitol, mannitol, or xylitol.

A lower limit of pH of the amino acid-containing solution of pH 3 or higher and pH 7 or lower is not particularly limited, except that the lower limit is pH of 3 or higher. The lower limit of pH of the solution may be appropriately selected according to the intended purpose. The lower limit of pH of the solution is preferably higher than pH 3, more preferably pH 3.25 or higher, yet more preferably pH 3.5 or higher, particularly preferably pH 4 or higher, and most preferably pH 4.25 or higher.

An upper limit of pH of the amino acid-containing solution of pH 3 or higher and pH 7 or lower is not particularly limited, except that the upper limit is pH of 7 or lower. The upper limit of pH of the solution may be appropriately selected according to the intended purpose. The upper limit of pH of the solution is preferably pH 6.5 or lower, more preferably pH 6 or lower, yet more preferably pH 5.5 or lower, particularly preferably pH 5 or lower, and most preferably pH 4.75 or lower.

In order for a buffer including an amino acid to be effective, pH of the buffer needs to be within a range of pKa±1, and preferably within a range of pKa±0.5 (Buffers for pH and Metal Ion Control D. D. Perrin and Boyd Dempsey 1974 London CHAPMAN AND HALL). For example, pKa of glycine is 2.35 and 9.77 (CALBIOCHEM Buffers A guide for the preparation and use of buffers in biological systems By Chandra Mohan, Ph. D. 2003 EMD Biosciences, Inc.). Therefore, a desired buffering capacity of glycine is obtained in the range of pH 1.85 to pH 2.85 and in the range of pH 9.27 to pH 10.27. When glycine is used for an eluent, use of a solution of pH 3 or higher and pH 7 or lower has not been expected. However, the present invention is made clear that an adeno-associated virus (AAV) bonded to an affinity carrier can be surprisingly efficiently separated (eluted) using, as an eluent, an amino acid (e.g., glycine)-containing solution of pH 3 or higher and pH 7 or lower.

### ——Adeno-Associated Virus (AAV)——

The adeno-associated virus is a virus that belongs to the Parvoviridae family and includes a linear single-strand DNA inside a capsid. As a serotype of the adeno-associated virus, a serotype 1 AAV (AAV1), a serotype 2 AAV (AAV2), a serotype 3 AAV (AAV3), a serotype 4 AAV (AAV4), a serotype 5 AAV (AAV5), a serotype 6 AAV (AAV6), a serotype 7 AAV (AAV7), a serotype 8 AAV (AAV8), a serotype 9 AAV (AAV9), a serotype 10 AAV (AAV10), and the like are known.

Among the above serotypes, a serotype 2 AAV (AAV2), a serotype 8 AAV (AAV8), or a serotype 9 AAV (AAV) is preferred, and a serotype 2 AAV (AAV2) or a serotype 8 AAV (AAV8) is more preferred.

The adeno-associated virus may be a capsid of a adeno-associated virus or a vector including an adeno-associated virus gene.

The vector including an adeno-associated virus gene may be a vector for therapy including a gene used for therapy. The gene used for therapy is not particularly limited, and may be appropriately selected according to the intended purpose.

### ——Affinity Carrier——

The affinity carrier includes a water-insoluble material and an antibody fixed to the water-insoluble material, and may further include other elements.

Specifically, in the affinity carrier, the water-insoluble material and molecules of the antibody may be directly bound with each other, or the water-insoluble material and each of the molecules of the antibody may be bound with each other via another element.

A density of the antibody molecules (ligand density) in the affinity carrier is not particularly limited, and may be appropriately selected according to the intended purpose. The density of the antibody molecules is preferably from 1 mg/mL to 20 mg/mL, more preferably from 1 mg/mL to 10 mg/mL, and yet more preferably from 2 mg/mL to 10 mg/mL.

The ligand density is measured in the following manner.

When a ligand is fixed to the water-insoluble material, a filtrate is collected. After passing the filtrate through a 0.2 μm-filter, absorbance is measured to calculate a ligand density of the ligand fixed to the water-insoluble material. The calculation of the ligand density is performed based on calibration curves prepared using absorbance of an antibody and the attenuation coefficient of BSA, and absorbance of the filtrate.

### ———Water-Insoluble Material———

The water-insoluble material is not particularly limited, and may be appropriately selected according to the intended purpose. Examples of the water-insoluble material include water-insoluble fibers, beads, a membrane (including hollow fibers), a monolith, and the like.

Among the above examples, water-insoluble fibers or beads are preferred.

### ————Water-Insoluble Fibers————

A lower limit of a thickness of the water-insoluble fibers is not particularly limited, and may be appropriately selected according to the intended purpose. In view of stability of production, the lower limit of the thickness of the water-insoluble fibers is preferably 0.08 mm or greater, more preferably 0.10 mm or greater, and yet more preferably 0.12 mm or greater.

An upper limit of the thickness of the water-insoluble fibers is not particularly limited, and may be appropriately selected according to the intended purpose. In view of a homogeneous basis weight, the lower limit of the thickness of the water-insoluble fibers is preferably 0.50 mm or less, more preferably 0.40 mm or less, and yet more preferably 0.30 mm or less.

A lower limit of a basis weight of the water-insoluble fibers is not particularly limited, and may be appropriately selected according to the intended purpose. In view of stability of production, the lower limit of the basis weight of the water-insoluble fibers is preferably 5 g/m² or greater, and more preferably 10 g/m² or greater.

An upper limit of the basis weight of the water-insoluble fibers is not particularly limited, and may be appropriately selected according to the intended purpose. In view of uniformity of the water-insoluble fibers, the upper limit of the basis weight of the water-insoluble fibers is preferably 100 g/m² or less, more preferably 90 g/m² or less, yet more preferably 80 g/m² or less, and particularly preferably 70 g/m² or less.

A lower limit of a bulk density of the water-insoluble fibers is not particularly limited, and may be appropriately selected according to the intended purpose. In view of minimization of change in the water-insoluble fiber structure after being loaded to a device, the lower limit of the bulk density of the water-insoluble fibers is preferably 50 kg/m³ or greater, more preferably 60 kg/m³ or greater, and yet more preferably 70 kg/m³ or greater.

An upper limit of the bulk density of the water-insoluble fibers is not particularly limited, and may be appropriately selected according to the intended purpose. In view of seeping, the upper limit of the bulk density of the water-insoluble fibers is preferably 400 kg/m³ or less, more preferably 350 kg/m³ or less, and yet more preferably 300 kg/m³ or less.

Note that, the bulk density is a value obtained by measuring a weight per 1 m³ of the water-insoluble fibers.

A shape of each of the water-insoluble fibers is not particularly limited, and may be appropriately selected according to the intended purpose. Examples of the shape include circles, squares, triangles, ovals, and the like.

Surfaces of the water-insoluble fibers may be modified by graft polymerization, polymer coating, chemical processing of alkali, acid, or the like, plasma processing, or the like.

The graft polymerization is not particularly limited, and may be appropriately selected according to the intended purpose. Examples of the graft polymerization include a graft polymerization method where electron-beam irradiation is performed, and the like.

The graft polymerization method where electron-beam irradiation is performed includes a pre-irradiation method and a simultaneous irradiation method. The pre-irradiation method is a method where an electron beam is applied to the water-insoluble fibers in advance to generate radicals, followed by applying a radically polymerizable compound to the water-insoluble fibers in which the radical species are generated, and then polymerization of a graft polymerizable compound is facilitated by postpolymerization. The simultaneous irradiation method is a method where, after applying a radically polymerizable compound to the water-insoluble fibers, an electron beam is applied to the resultant mixture to generate radicals, and then polymerization of a graft polymerizable compound is facilitated by postpolymerization. In the present invention, any of the pre-irradiation method or the simultaneous irradiation method can be applied.

In both the pre-irradiation method and the simultaneous irradiation method, a film is preferably laminated onto a surface of a bulk of the water-insoluble fibers during a period from a time after application of the radically polymerizable compound to the water-insoluble fibers to the completion of the postpolymerization. The film can prevent vaporization of the radically polymerizable compound to homogeneously initiate graft polymerization, and can block off oxygen to inhibit deactivation of radicals due to oxygen in the air. Moreover, in case of the simultaneous irradiation method, oxidation of the water-insoluble fibers caused by oxygen in the air is inhibited because a surface of the water-insoluble fibers is sealed with a film to shield the water-insoluble binders and the radically polymerizable compound from oxygen in the air during electron-beam irradiation.

The film is a polymer film having a thickness of 0.01 mm to 0.20 mm, and the film of a suitable thickness may be used according to penetrating power of an electron beam used.

A material of the film is not particularly limited, and may be appropriately selected according to the intended purpose. Examples of the material of the film include polyester-based materials, such as polyethylene terephthalate, polyolefin-based materials, and the like. Among the above-listed examples, polyethylene terephthalate is preferred. Particularly, in the case of the simultaneous irradiation method, a polyethylene terephthalate film, which has a low generation efficiency of polymer radicals with electron-beam irradiation and low oxygen permeability, is preferred.

In the pre-irradiation method, an electron beam is first applied to the water-insoluble fibers to generate radicals (polymer radicals, etc.) that initiate a polymerization reaction. As a temperature of the atmosphere during the electron-beam irradiation, the lower the temperature is, the higher the generation efficiency of radicals is. However, the temperature may be typical room temperature.

In the pre-irradiation method, irradiation conditions of the electron beam are not particularly limited, and may be appropriately selected according to the intended purpose. The irradiation conditions are appropriately adjusted according to a thickness of the water-insoluble fibers, a target grafting rate, or the like, preferably in a state where an oxygen concentration in the irradiation atmosphere is set at 300 ppm or less, at acceleration voltage of 100 kilovolts to 2,000 kilovolts (may be abbreviated as “kV” hereinafter), more preferably at 120 kV to 300 kV, and an electric current of 1 mA to 100 mA.

Moreover, an irradiation dose of the electron beam is appropriately determined considering a target grafting rate and potential deterioration of physical properties of the water-insoluble fibers. The irradiation dose is typically appropriately 10 kiloGray to appropriately 300 kiloGray (abbreviated as “kGy” hereinafter), and preferably 10 kGy to 200 kGy. The irradiation dose of lower than 10 kGy is not preferred because radicals used for an intended graft polymerization amount are not sufficiently generated. The irradiation dose of higher than 300 kGy is not preferred because deterioration in physical properties is caused due to scission of a main chain even when the water-insoluble fibers are formed of a polymer material having radiation resistance.

Note that, in the case of the pre-irradiation method, an irradiation atmosphere is preferably an inert gas atmosphere, such as of a nitrogen gas, but may be an air atmosphere. However, in the air atmosphere, there is a possibility that the water-insoluble fibers may be oxidized due to oxygen in the air.

Subsequently, a radically polymerizable compound is applied to the water-insoluble fibers which have been subjected to the electron-beam irradiation. For example, the water-insoluble fibers are immersed in a tank filled with a radically polymerizable compound solution in which dissolved oxygen is removed by blowing a nitrogen gas, or immersed and passed through the tank of the radically polymerizable compound solution to retain the water-insoluble fibers for a predetermined time, thereby sufficiently applying the radically polymerizable compound to the water-insoluble fibers.

Note that, the “immerse” in the present invention means that the water-insoluble fibers come into contact with the radically polymerizable compound solution. Therefore, as a method of applying the radically polymerizable compound to the water-insoluble fibers, various coating methods may be used. Among various coating methods, dip coating, comma direct coating, comma reverse coating, kiss coating, gravure coating, or the like is preferred because coating can be efficiently performed.

The water-insoluble fibers to which the radically polymerizable compound is applied are taken out from the solution tank. At this point, a surface of the water-insoluble fibers is preferably laminated with a film. In a case where the water-insoluble fibers are in the form of a sheet or fibers, for example, the water-insoluble fibers to which the radically polymerizable compound solution is applied are interposed between two sheets of films, and are closely adhered with the films. As the water-insoluble fibers to which the radically polymerizable compound solution is applied are closely adhered to the films, an application rate of the radically polymerizable compound solution is controlled to be constant, and the radically polymerizable compound can be homogeneously applied to the water-insoluble fibers. Moreover, as the water-insoluble fibers and the radically polymerizable compound are allowed to react in a space sealed by laminating the films, a graft reaction is further facilitated.

Note that, the “laminate” in the present invention means that the water-insoluble fibers and the film are in contact with each other.

The water-insoluble fibers taken out from the solution tank is retained in a postpolymerization tank of a predetermined temperature for a predetermined period so that graft polymerization of the radically polymerizable compound is facilitated (postpolymerization). At this process, a postpolymerization temperature is 0° C. to 130° C., and more preferably 40° C. to 70° C. As a result of this, a graft polymerization reaction between the water-insoluble fibers and the radically polymerizable compound is facilitated. Thereafter, the resultant fibers are washed and dried to thereby obtain grafted fibers. Note that, a postpolymerization atmosphere is preferably an inert gas atmosphere, such as of a nitrogen gas, but may be an air atmosphere when postpolymerization is performed in a state where the water-insoluble fibers are laminated with films.

In the case of the simultaneous irradiation method, moreover, the water-insoluble fibers are immersed in a tank of a radically polymerizable compound solution from which dissolved oxygen has been removed by blowing a nitrogen gas, or immersed in and passed through the tank to retain the water-insoluble fibers in the radically polymerizable compound solution for a predetermined time so that the radically polymerizable compound is sufficiently applied to the water-insoluble fibers. Thereafter, the water-insoluble fibers are taken out from the solution tank, and are irradiated with an electron beam. When the water-insoluble fibers are taken out from the solution tank, a film is preferably laminated on a surface of the water-insoluble fibers and an electron beam is applied in this state.

An acceleration voltage in the simultaneous irradiation method may be appropriately determined according to a type of a polymer material use, a total thickness of the water-insoluble fibers to which the radically polymerizable compound solution is applied and laminated film(s), and a target grafting rate. Typically, the acceleration voltage of approximately 100 kV to approximately 2,000 kV is appropriate. Moreover, an irradiation dose of the electron beam may be the same as in the pre-irradiation method. An atmosphere during the electron-beam irradiation is preferably an inert gas atmosphere, such as of nitrogen, of helium, and the like. In a case where a film is laminated to a surface of the water-insoluble fibers, the irradiation atmosphere does not affect graft polymerization, thus irradiation in the air is suitable considering cost efficiency.

Postpolymerization of the radically polymerizable compound is performed on the water-insoluble fibers, to which the electron-beam irradiation is performed, in the same manner as in the pre-irradiation method. Thereafter, washing and drying are performed to obtain grafted fibers. Note that, the postpolymerization atmosphere is preferably an inert gas atmosphere, such as of a nitrogen gas, also in the simultaneous irradiation method, but may be an air atmosphere when postpolymerization is performed in a state where a film is laminated to a surface of the water-insoluble fibers.

Moreover, the radically polymerizable compound for use in the present invention is a compound that forms a bond with a polymer radical generated at the water-insoluble fibers by the electron-beam irradiation. Specific examples of the radically polymerizable compound include, but are not limited to: acid group-containing unsaturated compounds, such as acrylic acid, methacrylic acid, itaconic acid, methacrylsulfonic acid, styrene sulfonic acid, and the like, and esters of the foregoing; unsaturated carboxylic acid amides, such as acrylamide, methacrylamide, and the like; unsaturated compounds including a glycidyl group, a hydroxyl group, an amino group, or a formyl group at a terminal; unsaturated organic phosphoric acid esters, such as vinyl phosphonate; methacrylic acid esters having a base, such as quaternary ammonium salts, tertiary ammonium salts, and the like; fluoroacrylate; acrylonitrile; and the like. The above examples may be used alone or in combination. Use of two or more radically polymerizable compounds can form composite graft fibers formed of a copolymer in which side chains includes two or more radically polymerizable compounds.

Among the above radically polymerizable compounds, an acrylic monomer is preferably used in the present invention in view of a grafting rate. In view of reactivity with a ligand including an amino group, a hydroxyl group, a thiol group, or the like, moreover, an acrylic monomer including a carboxyl group or an epoxy group at a terminal of a molecule thereof is preferred, and at least one selected from the group consisting of acrylic acid, methacrylic acid, and glycidyl methacrylate (abbreviated as “GMA” hereinafter) is more preferred.

The above radically polymerizable compound may be in a form of a dilute solution using water, an organic solvent such as lower alcohol, or a mixed solution of the foregoing as a solvent. A concentration of the radically polymerizable compound in the dilute solution may vary depending on a desired grafting rate, but the concentration of the radically polymerizable compound can be adjusted at 1% by volume to 70% by volume. Moreover, in a case where a radically polymerizable compound that tends to generate a homopolymer is used, a metal salt of copper or iron may be added to the dilute solution of the radically polymerizable compound to inhibit generation of a homopolymer.

A lower limit of a concentration of the radically polymerizable compound in the solution is not particularly limited, and may be appropriately selected according to the intended purpose. The lower limit of the concentration of the radically polymerizable compound is preferably 1% by weight or greater, more preferably 2.5% by weight or greater, yet more preferably 5% by weight or greater, and particularly preferably 10% by weight or greater.

An upper limit of a concentration of the radically polymerizable compound in the solution is not particularly limited, and may be appropriately selected according to the intended purpose. The upper limit of the concentration of the radically polymerizable compound is preferably 70% by weight or less, more preferably 60% by weight or less, yet more preferably 50% by weight or less, and particularly preferably 40% by weight or less.

Moreover, as the radically polymerizable compound solution includes a solvent, a grafting rate is improved. In this case, a concentration of an emulsifier in the solvent is preferably adjusted to the range of from 0.1% by weight to 5% by weight.

The emulsifier is not particularly limited, and may be appropriately selected according to the intended purpose. For example, the emulsifier is preferably Polysorbate. Examples of the Polysorbate include Polysorbate 20, 60, 65, 80, and the like. Among the above-listed examples, Polysorbate 20 that has high hydrophilicity is more preferred.

Note that, an inert gas, such as a nitrogen gas, is blown into the radically polymerizable compound solution in advance to remove dissolved oxygen therein.

A grafting rate of the graft polymerization reaction is not particularly limited, and may be appropriately selected according to the intended purpose. The grafting rate is preferably 50% or greater.

In the present invention, the “grafting rate” is a value calculated from a dry weight (W1) of a water-insoluble fiber before a graft reaction and a dry weight (W2) of a grafted fiber after a graft reaction as follows.

Grafting rate=[(W2−W1)/W1]×100(%)

A material of the water-insoluble fibers is not particularly limited, and may be appropriately selected according to the intended purpose. Examples of the material of the water-insoluble fibers include polyolefin-based materials, polypropylene, maleic anhydride-grafted polypropylene, modified polypropylene, polyethylene, cellulose, recycled cellulose, cellulose acetate, cellulose diacetate, cellulose triacetate, ethyl cellulose, acetic acid cellulose, polyethylene terephthalate (PET), polybutylene terephthalate (PBT), acrylic resins, polycarbonate, polyester-based materials, polyacrylonitrile, polyamide, polystyrene, brominated polystyrene, polyalkyl(meth)acrylate, polyvinyl chloride, polychloroprene, polyurethane, polyvinyl alcohol, polyvinyl acetate, polysulfone, polyether sulfone, polybutadiene, butadiene-acrylonitrile copolymers, styrene-butadiene copolymers, ethylene-vinyl alcohol copolymers, aramid, glass, nylon, rayon, and the like. The above-listed examples may be used alone or in combination. Among the above-listed example, polyolefin-based materials or cellulose-based materials are preferred, polyolefin-based materials are more preferred, polypropylene is yet more preferred in view of desirable reactivity for electron-beam graft polymerization.

A lower limit of a mean fiber diameter of the water-insoluble fibers is not particularly limited, and may be appropriately selected according to the intended purpose. The lower limit of the mean fiber diameter of the water-insoluble fibers is preferably 0.3 μm or greater, more preferably 0.4 μm or greater, and yet more preferably 0.5 μm or greater in view of desired tensile strength or in view of productivity.

An upper limit of a mean fiber diameter of the water-insoluble fibers is not particularly limited, and may be appropriately selected according to the intended purpose. The upper limit of the mean fiber diameter of the water-insoluble fibers is preferably 15 μm or less, more preferably 10 μm or less, yet more preferably 5 μm or less, and particularly preferably 3 μm or less in view of high purification capabilities. The water-insoluble fibers having the mean fiber diameter of greater than 15 μm are not preferred because of low purification capabilities.

A lower limit of a mean pore diameter of the water-insoluble fibers is not particularly limited, and may be appropriately selected according to the intended purpose. The lower limit of the mean pore diameter of the water-insoluble fibers is preferably 0.1 μm or greater, more preferably 1.0 μm or greater, and yet more preferably 1.5 μm or greater in view of desirable seeping or in view of productivity.

An upper limit of the mean pore diameter of the water-insoluble fibers is not particularly limited, and may be appropriately selected according to the intended purpose. The upper limit of the mean pore diameter of the water-insoluble fibers is preferably 50 μm or less, more preferably 30 μm or less, yet more preferably 20 μm or less, and particularly preferably 10 μm or less in view of high purification capabilities.

The water-insoluble fibers are not particularly limited, and may be appropriately selected according to the intended purpose. The water-insoluble fibers may be a nonwoven fabric, a woven fabric or a knitted fabric. In view of simplicity of production processes, a nonwoven fabric is preferred.

A production method of the nonwoven fabric is not particularly limited, and may be appropriately selected according to the intended purpose. Examples of the production method of the nonwoven fabric include a wet method, a dry method, a melt-blow method, an electrospinning method, a flash spinning method, a paper-making method, a spun-bonding method, a thermal bonding method, a chemical bonding method, a needle punching method, a spun-lacing method (hydroentangling method), a stitch-bonding method, and a steam jet method and the like. Among the above examples, a melt-blow method, an electrospinning method, a flash spinning method, a paper-making method, or the like is preferred in view of production of very fine fibers.

The melt-blow method is not particularly limited, and may be appropriately selected according to the intended purpose. Examples of the melt-blow method include a method where a melted thermoplastic resin is blown out from a melt-blowing die in a thread-like form with a high-temperature and high-speed air flow, and the resin stretched in a fibrous form is accumulated on a conveyor to cause entanglement and fusion of fibers, thereby obtaining a nonwoven fabric of self-adhesive ultrafine fibers without a binder, and the like. In the above method, a fiber diameter, basis weight, fiber orientation, and fiber dispersibility of the nonwoven fabric can be controlled by adjusting a viscosity of the resin, a melting temperature, an ejection amount, a temperature of the hot air, air pressure, a die to collector distance (DCD) (a distance from a spinneret to a surface of the conveyor). Furthermore, a thickness and a mean pore diameter of the nonwoven fabric can be controlled by hot pressing, tentering, or the like.

### ————Beads————

The beads are not particularly limited, and may be appropriately selected according to the intended purpose. The beads are preferably epoxidized beads or N-hydroxysuccinimide (NHS)-esterified beads.

A mean particle diameter of the beads is not particularly limited, and may be appropriately selected according to the intended purpose. For example, the mean particle diameter of the beads is 20 μm or greater and 1,000 μm or less based on a volume average particle diameter. When the volume average particle diameter is 20 μm or greater, a back pressure caused when the beads are loaded in a device can be kept low. When the volume average particle diameter is 1,000 μm or less, a surface area of the beads increases so that a large adsorption amount of a target compound can be retained. The volume average particle diameter is more preferably 30 μm or greater, and yet more preferably 40 μm or greater, and is more preferably 250 μm or less, yet more preferably 125 μm or less, yet further more preferably 100 μm or less, and yet further more preferably 60 μm or less.

The volume average particle diameter of the porous beads can be determined by measuring particle diameters of one hundred porous beads that are randomly selected. A particle diameter of each porous bead can be measured by taking a micrograph of each porous bead, which is stored as an electronic data, and measuring the micrograph of the stored electronic data using particle size analysis software (e.g., “Image-Pro Plus” manufactured by Media Cybernetics Inc.). The porous beads are preferably crosslinked with a polyfunctional compound according to a commonly known method for improving the strength or the like.

A material of the beads is not particularly limited, and may be appropriately selected according to the intended purpose. Examples of the material of the beads include: polysaccharides, such as cellulose, agarose, dextran, starch, pullulan, chitosan, chitin, and the like; synthetic polymers and crosslinked products thereof, such as poly(meth)acrylic acid, poly(meth)acrylate, polyacrylamide, polyvinyl alcohol, and the like; and glass, such as silica glass, borosilicate glass, optical glass, soda-lime glass, and the like.

Moreover, a surface of a base formed of a synthetic polymer having no functional group, such as polystyrene, a styrene-divinyl benzene copolymer, or the like, may be coated with a polymer material having a reactive functional group, such as a hydroxyl group. Examples of the polymer material for coating include hydroxyethyl methacrylate, graft copolymers, such as a copolymer of a monomer having a polyethylene oxide chain and another polymerizable monomer having a reactive functional group, and the like. The above-listed examples may be used alone or in combination.

Examples of a commercial product of the beads include GCL2000 that is a porous cellulose gel, Sephacryl (registered trademark) S-1000 in which allyl dextran and methylene bisacrylamide are crosslinked with a covalent bond, Toyopearl (registered trademark) that is a methacrylate-based carrier, Sepharose CL4B that is an agarose-based crosslinked carrier, Cellufine (registered trademark) that is a cellulose-based crosslinked carrier, POROS (registered trademark) 500H that is a carrier in which a styrene-divinyl benzene copolymer is coated with a polymer material, and the like. Among the above examples, POROS (registered trademark) 500H is preferred in view of a desired particle diameter range and the presence of a reactive functional group that facilitates a modification reaction.

### ————Monolith————

The monolith is not particularly limited, and may be appropriately selected according to the intended purpose. The monolith is preferably a carboxyimidazole activated monolith.

A mean pore diameter of the monolith is not particularly limited, and may be appropriately selected according to the intended purpose. In view of desirable seeping or productivity, the mean pore diameter of the monolith is preferably 0.1 μm or greater, more preferably 1.0 μm or greater, and yet more preferably 1.5 μm or greater.

An upper value of the mean pore diameter of the monolith is not particularly limited, and may be appropriately selected according to the intended purpose. In view of high purification capabilities, the upper value of the mean pore diameter of the monolith is preferably 50 μm or less, more preferably 30 μm or less, yet more preferably 20 μm or less, and particularly preferably 10 μm or less.

The monolith is formed using a polyvinyl monomer and a monovinyl monomer. Kinds of the polyvinyl monomer and the monovinyl monomer are not particularly limited, and may be appropriately selected according to the intended purpose. Examples of the polyvinyl monomer include divinyl benzene, divinyl naphthalene, divinyl pyridine, alkylene dimethacrylates, hydroxyalkylene dimethacrylates, hydroxyalkylene diacrylates, oligoethylene glycol diacrylates, polyvinyl carboxylic acids, vinyl ethers, pentaerythritol di-, tri-, or tetramethacrylate or pentaerythritol di-, tri-, or tetraacrylate, trimethylolpropane trimethylacrylate or trimethylolpropane acrylate, alkylene bisacrylamides or alkylene bismethacrylamides, ethylene dimethacrylate, and mixtures of the foregoing. Examples of the monovinyl monomer include styrene, ring-substituted styrene (with proviso that a substituent includes a chloromethyl group, an alkyl group having 18 or less carbon atoms, a hydroxide group, a t-butyloxycarbonyl group, a halogen group, a nitro group, an amino group, a protecting hydroxyl group, or an amino group), vinyl naphthalene, acrylic acid esters, methacrylic acid esters, glycidyl methacrylate, vinyl acetate, pyrrolidone, and mixtures of the foregoing.

Examples of a commercial product thereof include CIMmic (registered trademark) CDI-0.1 Disk (Carboxy imidazole) formed of ethylene dimethacrylate and glycidyl methacrylate (manufactured by Sartorius AG) and the like.

### ————Antibody————

The antibody is not particularly limited, except that the antibody is an antibody bonded to an adeno-associated virus (AAV). The antibody may be appropriately selected according to the intended purpose. The antibody may be a whole antibody or an antibody fragment, and is preferably an antibody fragment.

The antibody fragment is not particularly limited, and may be appropriately selected according to the intended purpose. Examples of the antibody fragment include variable regions (VHH) of heavy-chain antibodies derived from camelids, variable regions (V-NAR) of heavy-chain antibodies derived from fish, Fab, Fab′, F(ab′)₂, single chain antibodies (scFv), diabodies, triabodies, minibodies, and the like. Among the above examples, a variable region (VHH) of a heavy-chain antibody derived from camelids or a single-strand antibody (scFv) is preferred in view of stability and production efficiency.

The variable region (VHH) of a heavy-chain antibody derived from camelids is not particularly limited, and may be appropriately selected according to the intended purpose. Examples of the variable region (VHH) include those purified from serum from a camelid immunized with the adsorption target, those produced by expressing a VHH gene in a host cell, those chemically synthesized based on an amino acid sequence, and the like.

The camelids are not particularly limited, and may be appropriately selected according to the intended purpose. Examples of the camelids include Bactrian camels, dromedaries, llamas, alpacas, vicuna, guanaco, and the like.

The host cell used for expressing the VHH gene is not particularly limited, and may be appropriately selected according to the intended purpose. Examples of the host cell include bacteria such as Escherichia coli, fungi such as yeast, animal cells, plant cells, and the like.

A method of immunizing the camelid with the adsorption target is not particularly limited, and may be appropriately selected according to the intended purpose. Examples of the method include the method disclosed in International Publication No. WO 2020/067418.

A method of producing the variable region (VHH) by expressing the VHH gene in the host cell is not particularly limited, and may be appropriately selected according to the intended purpose. Examples of the method include methods disclosed in International Publication No. WO 2020/067418, Japanese Unexamined Patent Application Publication No. 2015-119637, and the like.

The single-strand antibody (scFv) is obtained by linking VH and VL of an antibody. In the scFv, VH and VL are linked via a linker, preferably a peptide linker (Proc. Natl. Acad. Sci. U.S.A 1988 85:5879). The peptide linker is not particularly limited. For example, a suitable single-strand peptide composed of approximately 3 residues to approximately 25 residues can be used as the linker.

The scFv is not particularly limited, and may be appropriately selected according to the intended purpose. Examples of the scFv include those produced by expressing an scFv gene in a host cell, those chemically synthesized based on an amino acid sequence, and the like.

The host cell used for expressing the scFv gene is not particularly limited, and may be appropriately selected according to the intended purpose. Examples of the host cell include bacteria such as Escherichia coli, fungi such as yeast, animal cells, plant cells, and the like.

The antibody fragment preferably includes a single domain antibody.

The single domain antibody is not particularly limited, and may be appropriately selected according to the intended purpose. The single domain antibody is preferably a single domain antibody including a variable region (VHH) of a heavy-chain antibody derived from camelids.

The antibody fragment may be chimeric or humanized.

A molecular weight of the antibody fragment is not particularly limited, and may be appropriately selected according to the intended purpose. The molecular weight of the antibody fragment is preferably 130,000 or less, more preferably 100,000 or less, yet more preferably 50,000 or less, particularly preferably 30,000 or less, and the most preferably 20,000 or less.

### ———Other Elements———

The above other elements are not particularly limited, and may be appropriately selected according to the intended purpose. Examples of the above other elements include spacers, and the like.

The affinity carrier is not particularly limited, and may be appropriately selected according to the intended purpose. As the affinity carrier, an affinity carrier produced by a method known in the related art may be used, or a commercial product of the affinity carrier may be used.

Examples of the commercial product of the affinity carrier include POROS (registered trademark) CaptureSelect (registered trademark) AAV8, AAV9, AAVX (manufactured by Thermo Fisher Scientific Inc.), CaptoAVB, AVB Sepharose, and the like.

The bonding is not particularly limited, and may be appropriately selected according to the intended purpose. For example, bonding or adsorption between the affinity carrier and the adeno-associated virus (AAV) can be achieved by allowing the affinity carrier and the adeno-associated virus (AAV) to be in contact with each other.

The contact is not particularly limited, and may be appropriately selected according to the intended purpose. Examples of a method for the contact include a method where the affinity carrier and the adeno-associated virus (AAV) are mixed, a method where a solution including the adeno-associated virus (AAV) is passed through a column loaded with the affinity carrier, and the like.

A material of the column is not particularly limited, and may be appropriately selected according to the intended purpose. Examples of the material of the column include glass, resins, such as polypropylene, acrylics, and the like, and metals, such as stainless steel and the like.

The separation is not particularly limited, and may be appropriately selected according to the intended purpose. Examples of a method for the separation include a method where the affinity carrier to which the adeno-associated virus (AAV) is bonded and the amino acid-containing solution of pH 3 or higher and pH 7 or lower are mixed, a method where the amino acid-containing solution of pH 3 or higher and pH 7 or lower is passed through a column loaded with the affinity carrier to which the adeno-associated virus (AAV) is bonded, and the like.

By the separation step, the adeno-associated virus (AAV) is separated, dissociated, or eluted from the affinity carrier.

Gene transfer efficiency of the adeno-associated virus (AAV), which is separated by the separation step, 24 hours after the separation is preferably 110% or greater, more preferably 150% or greater, more preferably 200% or greater, and particularly preferably 250$ or greater, relative to gene transfer efficiency of an adeno-associated virus (AAV), which is separated using a solution of pH 2.6, 24 hours after the separation.

### —Other Steps—

Examples of the above other steps include an AAV production step before the separation step, a neutralization step after the separation step, and the like.

### ——AAV Production Step Before the Separation Step——

The AAV production step before the separation step is not particularly limited, and may be appropriately selected according to the intended purpose. Examples of the AAV production step include a method where an AAV gene is expressed in a host cell to acquire a cell culture solution including the AAV, and the like.

The AAV production step may include a process of separating the cells.

The process of separating the cells is not particularly limited. Examples of the process include cell lysis, nucleic acid decomposition processing, and the like. Thereafter, clarification by membrane separation or centrifugal separation, removal of impurities or concentration by ultrafiltration, or filtration with a sterilizing filter may be performed.

Moreover, a step of processing full particles (particles in which a gene is included in the virus) and empty particles (particles in which a gene is not included in the virus) may be included after chromatography purification.

A step of separating the full particles and the empty particles is not particularly limited. Examples of the step include density gradient separation, ultracentrifugation, ion exchange chromatography, and the like.

Furthermore, a desalination concentration treatment may be included.

The desalination concentration treatment is not particularly limited. Examples of the desalination concentration treatment include ultrafiltration.

——Neutralization Step after the Separation Step——

The neutralization step is not particularly limited, and may be appropriately selected according to the intended purpose. Examples of the neutralization step include a method of neutralizing using a 1 M trishydroxymethylaminomethane (Tris) buffer, and the like.

### (Method of Purifying Adeno-Associated Virus (AAV))

The method of purifying an adeno-associated virus (AAV) includes a separation step and may further include other steps.

The separation step and other steps are as described in the above method of producing an adeno-associated virus (AAV).

### (Method of Inhibiting Reduction in Infectious Titer of Adeno-Associated Virus (AAV))

The method of inhibiting a reduction in an infectious titer of the adeno-associated virus (AAV) includes a separation step, and may further include other steps.

The separation step and other steps are as described in the above method of producing an adeno-associated virus (AAV).

As described above, in the present invention, gene transfer efficiency of the adeno-associated virus (AAV), which is separated by the separation step, 24 hours after the separation is preferably 110% or greater, more preferably 150% or greater, more preferably 200% or greater, and particularly preferably 250% or greater, relative to gene transfer efficiency of an adeno-associated virus (AAV), which is separated using a solution of pH 2.6, 24 hours after the separation.

Therefore, the method of the present invention including the separation step can inhibit a reduction in the infectious titer of the adeno-associated virus (AAV).

(Eluent for Separating Adeno-Associated Virus (AAV) Bonded to Affinity Carrier from Affinity Carrier)

The eluent for separating an adeno-associated virus (AAV) bonded to an affinity carrier from the affinity carrier includes an amino acid-containing solution of pH 3 or higher and pH 7 or lower. The eluent may further include other components.

The amino acid-containing solution of pH 3 or higher and ph 7 or lower is as described in the above method of producing an adeno-associated virus (AAV).

### EXAMPLES

Examples of the present invention will be described hereinafter, but the present invention is not limited by Examples in any way.

A detailed method associated with a recombinant DNA technology and the like used in Examples below are described in the following: Molecular Cloning 2nd Edition (Cold Spring Harbor Laboratory Press, 1989), Current Protocols in Molecular Biology (Green Publishing Associates and Willey-Interscience).

For cloning, In-fusion HD-cloning Kit (produced by Takara Bio Inc.) was used, and a reaction was performed under the reaction conditions described in the attached manual.

### (Production Example 1) Production of Adeno-Associated Virus (AAV8) Using Animal Cells

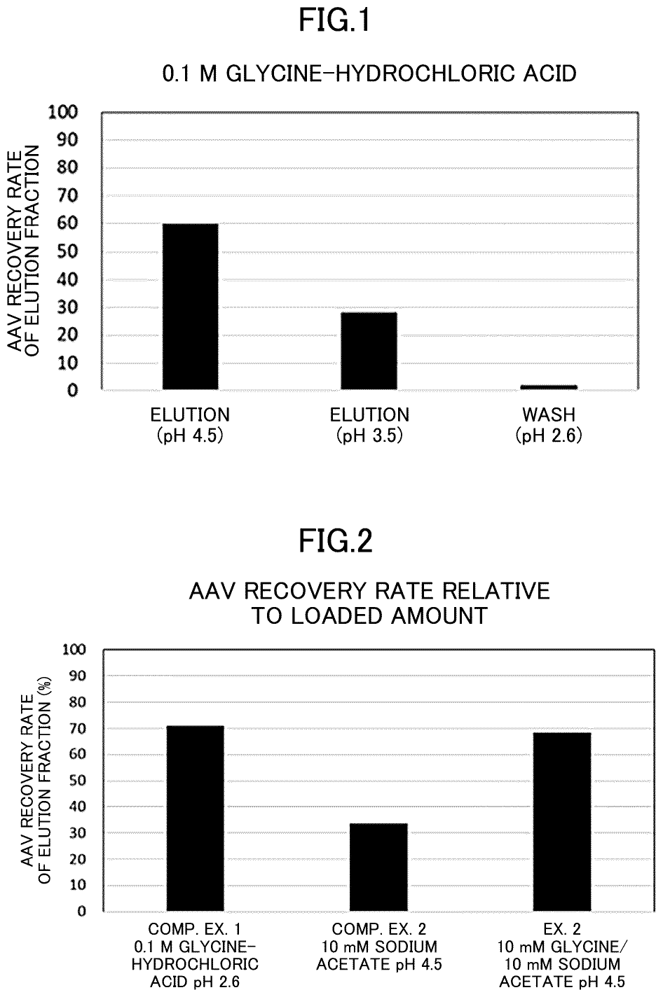

As plasmids for producing AAV8 expressing VENUS (GenBank: ACQ43955.1), which was a variant of a fluorescent protein GFP, a plasmid in which the VENUS was inserted into a pAAV-CMV Vector of an AAV vector production kit (“AAVpro (registered trademark) Helper Free System” produced by Takara Bio Inc.), and a plasmid in which a sequence information of Japanese Patent No. 4810062 (the base position of 2,121 to 4,337 in FIGS. 1B and 1C) was inserted into an AAV2 Cap region of a pRC2-mi342 Vector were prepared.

The cultured HEK293 cells were transfected with the produced plasmids, and the pHelper vector using a transfection reagent (“Polyethylenimine MAX” manufactured by Polysciences, MW: 40,000), to thereby produce AAV8. After completing the culturing, the cells were detached, and the cell culture solution was collected.

### (Production Example 2) Preparation of Pre-Treated AAV8 Liquid

The AAV8 cell culture solution obtained in Production Example 1 was suspended in Dulbecco's phosphate-buffered physiological saline (manufactured by Sigma-Aldrich, abbreviated as “PBS” hereinafter) including 0.1% Triton X-100, and the resultant suspension was stirred for 20 minutes on ice to disrupt the cells. To the obtained disrupted cell solution, a 1 M magnesium chloride aqueous solution in an amount of 0.75% (v/v) and a 250 kU/mL KANEKA Endonuclease (manufactured by KANEKA CORPORATION) in an amount of 0.1% (v/v) were added. The resultant mixture was left to stand for 30 minutes at 37° C. to decompose nucleic acids derived from the cells. After the reaction, a 0.5 M EDTA solution in an amount of 1.5% (v/v) relative to the reaction solution was added to the reaction solution, followed by centrifuge to separate the mixture into a supernatant and a sediment. To the supernatant, 8% (final concentration) polyethylene glycol and 0.5 M (final concentration) NaCl were added. After leaving the resultant mixture to stand overnight, the mixture was subjected to centrifuge, followed by re-suspending in PBS, to thereby recover AAV8. To the above sediment, 0.1% (final concentration) triton was added, and the resultant mixture was processed on ice for 20 minutes to disrupt the cells. Thereafter, an endonuclease treatment and inactivation treatment were performed, and AAV8 was recovered as a centrifuge supernatant. The AAV8 recovered from the supernatant and the AAV8 recovered from the sediment were mixed to thereby prepare a pre-treated AAV8 liquid.

### (Production Example 3) Preparation of Clarified AAV8 Liquid

Triton X-100 was added to the AAV8 cell culture solution obtained in Production Example 1 so that a final concentration was to be 0.1%. The resultant mixture was stirred for 30 minutes on ice to disrupt the cells. To the obtained disrupted cell solution, a 1 M magnesium chloride aqueous solution in an amount of 0.75% (v/v) and KANEKA Endonuclease (manufactured by KANEKA CORPORATION) whose final concentration was to be 50 U/mL were added. The resultant mixture was left to stand for 30 minutes at 37° C. to decompose nucleic acids derived from the cells. After the reaction, a 0.5 M EDTA solution in an amount of 1.5% (v/v) relative to the reaction solution was added to the reaction solution to terminate the reaction. Thereafter, the degraded nucleic acid solution was clarified with a depth filter of Supracap 50 capsule with V100P (manufactured by PALL) using AKTA Flux (registered trademark) s (manufactured by Cytiva) to thereby prepare a clarified AAV8 liquid.

### (Production Example 4) Preparation of Purified AAV8 Liquid

With reference to the non-patent document (Molecular Therapy. Methods & Clinical development 2020 19:362-373, December 11), the pre-treated AAV8 liquid obtained in Production Example 2 was purified by affinity chromatography. Tricorn (registered trademark) 5/50 (manufactured by Cytiva) was packed with POROS (registered trademark) CaptureSelect (registered trademark) AAV8 (manufactured by Thermo Fisher Scientific Inc.) to thereby prepare a column for affinity exchange purification.

Liquids A to E below were prepared, and were each passed through a 0.2 μm filter before use.
Liquid A: 1×TD buffer (137 mM sodium chloride, 8.1 mM disodium hydrogen phosphate, 2.68 mM potassium chloride, and 1.47 mM potassium dihydrogen phosphate)
Liquid B: 0.1 M glycine-hydrochloric acid of pH 2.6
Liquid C: 0.01 M sodium hydroxide
Liquid D: PAB solution (120 mM phosphoric acid, 167 mM acetic acid, and 2.2 mM benzyl alcohol)
Liquid E: Solution obtained by diluting the pre-treated AAV8 liquid with Liquid A to adjust to pH 7.4

### Production Method of 0.1 M Glycine-Hydrochloric Acid Solution of pH 2.6

1. Glycine was weighed out in a beaker to be 0.1 M, and was dissolved in approximately 80% of a total amount of ultrapure water to be used.
2. Hydrochloric acid was added to the above solution to adjust to pH 2.6.
3. The remaining ultrapure water was added to the solution to dilute to the gauge level.

The above column was connected to AKTA (registered trademark) Avant 25 (manufactured by Cytiva), and was equilibrated with Liquid A. Thereafter, Liquid E was passed through to allow the column carrier to retain AAV thereon, followed by washing with Liquid A, and AAV was eluted with Liquid B. After completing the elution, the column was washed with Liquid C and Liquid D.

The AAV8 of the purified elution fraction was neutralized with a 1 M Tris buffer, followed by determining an amount of AAV in the liquid through quantitative PCR (qPCR) using QuantStudio3 real-time PCR system (manufactured by Thermo Fisher Scientific Inc.). For the determination of the AAV8 amount, AAVpro Titration Kit (for Real Time PCR) Ver. 2 (registered trademark) was used.

### (Production Example 5) Production of Adeno-Associated Virus (AAV2) Using Animal Cells

As a plasmid for producing AAV2 expressing VENUS (GenBank: ACQ43955.1), which was a variant of a fluorescent protein GFP, a plasmid in which the VENUS was inserted into a pAAV-CMV Vector of an AAV vector production kit (“AAVpro (registered trademark) Helper Free System” produced by Takara Bio Inc.) was produced.

The cultured HEK293 cells were transfected with the produced plasmid, a pHelper vector, and a pRC2-mi342 vector using a transfection reagent (“Polyethylenimine MAX” manufactured by Polysciences, MW: 40,000), to thereby produce AAV2. After completing the culturing, the cells were scraped, and the cell culture solution was collected.

### (Production Example 6) Preparation of Pre-Treated AAV2 Liquid

The AAV2 cell culture solution obtained in Production Example 5 was suspended in Dulbecco's phosphate-buffered physiological saline (manufactured by Sigma-Aldrich, abbreviated as “PBS” hereinafter) including 0.1% Triton X-100, and the resultant suspension was stirred for 20 minutes on ice to disrupt the cells. To the obtained disrupted cell solution, a 1 M magnesium chloride aqueous solution in an amount of 0.75% (v/v) and a 250 kU/mL KANEKA Endonuclease (manufactured by KANEKA CORPORATION) in an amount of 0.1% (v/v) were added. The resultant mixture was left to stand for 30 minutes at 37° C. to decompose nucleic acids derived from the cells. After the reaction, a 0.5 M EDTA solution in an amount of 1.5% (v/v) relative to the reaction solution was added to the reaction solution, followed by centrifuge to separate the mixture into a supernatant and a sediment. To the supernatant, 8% (final concentration) polyethylene glycol and 0.5 M (final concentration) NaCl were added. After leaving the resultant mixture to stand overnight, the mixture was subjected to centrifuge, followed by re-suspending in PBS, to thereby recover AAV2. To the above sediment, 0.1% (final concentration) triton was added, and the resultant mixture was processed on ice for 20 minutes to disrupt the cells. Thereafter, an endonuclease treatment and inactivation treatment were performed, and AAV2 was recovered as a centrifuge supernatant. The AAV2 recovered from the supernatant and the AAV2 recovered from the sediment were mixed to thereby prepare a pre-treated AAV2 liquid.

### (Production Example 7) Preparation of Clarified AAV2 Liquid

Triton X-100 was added to the AAV2 cell culture solution obtained in Production Example 5 so that a final concentration was to be 0.1%. The resultant mixture was stirred for 30 minutes on ice to disrupt the cells. To the obtained disrupted cell solution, a 1 M magnesium chloride aqueous solution in an amount of 0.75% (v/v) and KANEKA Endonuclease (manufactured by KANEKA CORPORATION) whose final concentration was to be 50 U/mL were added. The resultant mixture was left to stand for 30 minutes at 37° C. to decompose nucleic acids derived from the cells. After the reaction, a 0.5 M EDTA solution in an amount of 1.5% (v/v) relative to the reaction solution was added to the reaction solution to terminate the reaction. Thereafter, the degraded nucleic acid solution was clarified with a depth filter of Supracap 50 capsule with V100P (manufactured by PALL) using AKTA Flux (registered trademark) s (manufactured by Cytiva) to thereby prepare a clarified AAV2 liquid.

Elution Evaluation of AAV8 with Amino Acid-Containing Solution

### Example 1

Whether AAV8 could be eluted from a column with an amino acid-containing solution (a glycine solution of a weak acid region) was evaluated using the purified AAV8 liquid prepared in Production Example 4.

Tricorn (registered trademark) 5/50 (manufactured by Cytiva) was packed with POROS (registered trademark) CaptureSelect (registered trademark) AAV8 (manufactured by Thermo Fisher Scientific Inc.) to thereby prepare a column for affinity exchange purification.

Liquids A to G below were prepared, and were each passed through a 0.2 μm filter before use.

The purified AAV8 was loaded onto the POROS (registered trademark) Capture Select (registered trademark) AAV8 (manufactured by Thermo Fisher Scientific Inc.), and an amount of AAV detected from the elution fraction was evaluated.
Liquid A: 1×TD buffer (137 mM sodium chloride, 8.1 mM disodium hydrogen phosphate, 2.68 mM potassium chloride, and 1.47 mM potassium dihydrogen phosphate)
Liquid B: 0.1 M glycine-hydrochloric acid solution of pH 4.5
Liquid C: 0.1 M glycine-hydrochloric acid solution of pH 3.5
Liquid D: 0.1 M glycine-hydrochloric acid solution of pH 2.6
Liquid E: 0.01 M sodium hydroxide solution
Liquid F: PAB solution (120 mM phosphoric acid, 167 mM acetic acid, and 2.2 mM benzyl alcohol)
Liquid G: Solution obtained by diluting the purified AAV8 liquid with Liquid A

### Production Method of 0.1 M Glycine-Hydrochloric Acid Solution of pH 4.5, 0.1 M Glycine-Hydrochloric Acid Solution of pH 3.5, or 0.1 M Glycine-Hydrochloric Acid Solution of pH 2.6

1. Glycine was weighed out in a beaker to be 0.1 M, and was dissolved in approximately 80% of a total amount of ultrapure water to be used.
2. Hydrochloric acid was added to the above solution to adjust to an intended pH (pH 4.5, pH 3.5, or pH 2.6).
3. The remaining ultrapure water was added to the solution to dilute to the gauge level.

The above column was connected to AKTA (registered trademark) Avant 25 (manufactured by Cytiva), and was equilibrated with Liquid A. Thereafter, Liquid G was passed through to allow the column carrier to retain AAV8 thereon, followed by washing with Liquid A, and AAV8 was eluted with Liquid B and Liquid C in this order. The column was washed with Liquid D, Liquid E, and Liquid F.

The AAV8 of the purified elution fractions (Liquid B and Liquid C), and AAV8 of the wash fraction (strip fraction) (Liquid D) was neutralized with a 1 M Tris buffer, an amount of the AAV8 in each liquid was determined through quantitative PCR (qPCR) using QuantStudio3 real-time PCR system (manufactured by Thermo Fisher Scientific Inc.) in the same manner as in Production Example 4. For the determination of the AAV8 amount, AAVpro Titration Kit (for Real Time PCR) Ver. 2 (registered trademark) was used. The results are presented in FIG. 1.

It was found from the results of FIG. 1 that AAV was eluted with Liquid B (0.1 M glycine-hydrochloric acid solution of pH 4.5) or Liquid C (0.1 M glycine-hydrochloric acid solution of pH 3.5).

Purification Evaluation of AAV with Amino Acid-Containing Solution

### Example 2

Whether AAV8 could be eluted from a column with an amino acid-containing solution was evaluated using the clarified AAV8 liquid prepared in Production Example 3.

Tricorn (registered trademark) 5/50 (manufactured by Cytiva) was packed with POROS (registered trademark) CaptureSelect (registered trademark) AAV8 (manufactured by Thermo Fisher Scientific Inc.) to thereby prepare a column for affinity exchange purification.

Liquids A to F below were prepared, and were each passed through a 0.2 μm filter before use.

The purified AAV8 was loaded onto the POROS (registered trademark) Capture Select (registered trademark) AAV8 (manufactured by Thermo Fisher Scientific Inc.), and an amount of AAV detected from the elution fraction was evaluated.
Liquid A: 1×TD buffer (137 mM sodium chloride, 8.1 mM disodium hydrogen phosphate, 2.68 mM potassium chloride, and 1.47 mM potassium dihydrogen phosphate)
Liquid B: 10 mM glycine/10 mM sodium acetate solution of pH 4.5
Liquid C: 0.1 M glycine-hydrochloric acid solution of pH 2.6
Liquid D: 0.01 M sodium hydroxide solution
Liquid E: PAB solution (120 mM phosphoric acid, 167 mM acetic acid, and 2.2 mM benzyl alcohol)
Liquid F: clarified AAV8 liquid

The 0.1 M glycine-hydrochloric acid solution of pH 2.6 was produced by the method described in Production Example 4.

### Production Method of 10 mM Glycine/10 mM Sodium Acetate Solution of pH 4.5

1. A 10 mM glycine/10 mM acetic acid solution and a 10 mM glycine/10 mM sodium acetate solution were produced.
2. The above two solutions were mixed to adjust to pH 4.5.

The above column was connected to AKTA (registered trademark) Avant 25 (manufactured by Cytiva), and equilibrated with Liquid A. Thereafter, Liquid F was passed through to allow the column carrier to retain AAV thereon, followed by washing with Liquid A, and AAV was eluted with Liquid B. The column was washed with Liquid C, Liquid D, and Liquid E.

The AAV of the purified elution fraction (Liquid B) was neutralized in a 1 M Tris buffer, followed by determining an amount of AAV8 in the liquid through quantitative PCR (qPCR) using QuantStudio3 real-time PCR system (manufactured by Thermo Fisher Scientific Inc.) in the same manner as in Production Example 4. For the determination of the AAV8 amount, AAVpro Titration Kit (for Real Time PCR) Ver. 2 (registered trademark) was used. The result is presented in FIG. 2 (right).

### Comparative Example 1

An amount of AAV in the purified elution fraction (Liquid B) was determined in the same manner as in Example 2, except that as Liquid B, a 0.1 M glycine-hydrochloric acid solution of pH 2.6 was used instead of the 10 mM glycine/10 mM sodium acetate solution of pH 4.5. The result is presented in FIG. 2 (left).

The 0.1 M glycine-hydrochloric acid solution of pH 2.6 was produced by the method described in Production Example 4.

### Comparative Example 2

An amount of AAV in the purified elution fraction (Liquid B) was determined in the same manner as in Example 2, except that as Liquid B, a 10 mM sodium acetate solution of pH 4.5 was used instead of the 10 mM glycine/10 mM sodium acetate solution of pH 4.5. The result is presented in FIG. 2 (center).

### Production Method of 10 mM Sodium Acetate Solution of pH 4.5

1. A 10 mM acetic acid solution and a 10 mM sodium acetate solution were produced.
2. The above two solutions were mixed to adjust to pH 4.5.

It was found from the results of FIG. 2 that, when the elution was performed with the amino acid-containing solution of pH 3 or higher and pH 7 or lower (Example 2), the recovery rate of the purified AAV8 from the elution fraction increased compared to the case where no amino acid was added (Comparative Example 2), and the increased rate was comparable to the case where the solution of lower than pH 3 was used (Comparative Example 1).

Purification Evaluation 2 of AAV8 with Amino Acid-Containing Solution

### Example 3

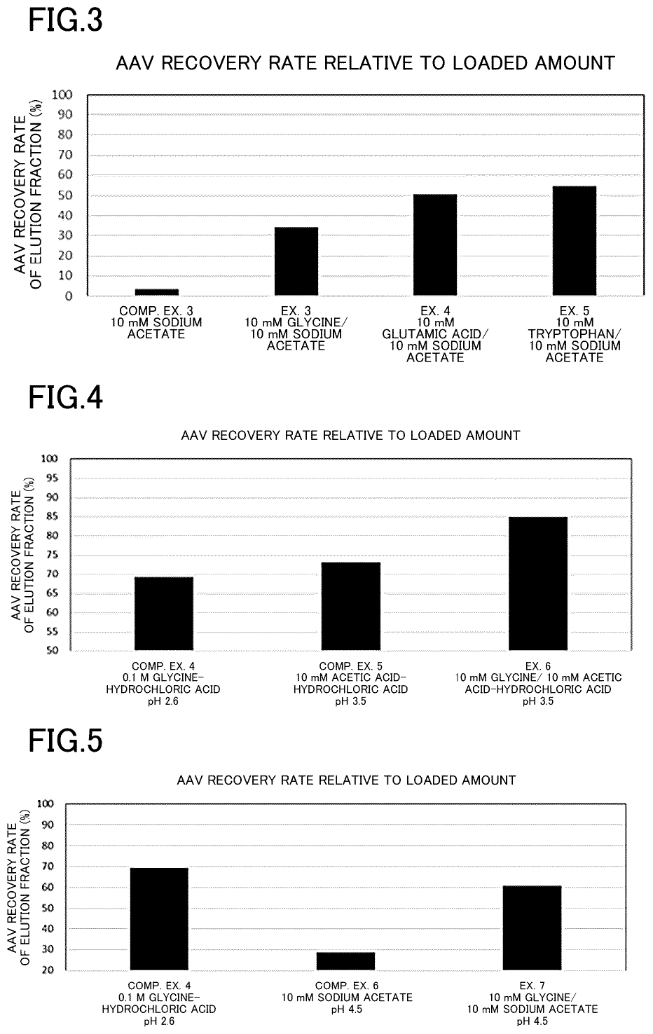

An amount of AAV in the purified elution fraction (Liquid B) was determined in the same manner as in Example 2. The result is presented in FIG. 3 (second from the left).

### Example 4

An amount of AAV in the purified elution fraction (Liquid B) was determined in the same manner as in Example 2, except that as Liquid B, a 10 mM glutamic acid/10 mM sodium acetate solution of pH 4.5 was used instead of the 10 mM glycine/10 mM sodium acetate solution of pH 4.5. The result is presented in FIG. 3 (second from the right).

### Production Method of 10 mM Glutamic Acid/10 mM Sodium Acetate Solution of pH 4.5

1. A 10 mM glutamic acid/10 mM acetic acid solution and a 10 mM glutamic acid/10 mM sodium acetate solution were produced.
2. The above two solutions were mixed to adjust to pH 4.5.

### Example 5

An amount of AAV in the purified elution fraction (Liquid B) was determined in the same manner as in Example 2, except that as Liquid B, a 10 mM tryptophan/10 mM sodium acetate solution of pH 4.5 was used instead of the 10 mM glycine/10 mM sodium acetate solution of pH 4.5. The result is presented in FIG. 3 (right).

### Production Method of 10 mM Tryptophan/10 mM Sodium Acetate Solution of pH 4.5

1. A 10 mM tryptophan/10 mM acetic acid solution and a 10 mM tryptophan/10 mM sodium acetate solution were produced.
2. The above two solutions were mixed to adjust to pH 4.5.

### Comparative Example 3

An amount of AAV in the purified elution fraction (Liquid B) was determined in the same manner as in Comparative Example 2. The result is presented in FIG. 3 (left).

It was confirmed from the results of FIG. 3 that the recovery rate was increased by adding, as the amino acid other than glycine, glutamic acid that was a typical example of an acidic amino acid, or tryptophan that was a typical example of an aromatic amino acid.

Purified Evaluation 3 of AAV8 with Amino Acid-Containing Solution

### Example 6

An amount of AAV in the purified elution fraction (Liquid B) was determined in the same manner as in Example 2, except that as Liquid B, a 10 mM glycine/10 mM acetic acid solution of pH 3.5 was used instead of the 10 mM glycine/10 mM sodium acetate solution of pH 4.5. The result is presented in FIG. 4 (right).

### Production Method of 10 mM Glycine/10 mM Acetic Acid Solution of pH 3.5

1. Glycine and acetic acid were weighed out in a beaker to be 10 mM, and was dissolved in approximately 80% of a total amount of ultrapure water to be used.
2. Hydrochloric acid was added to the above solution to adjust to pH 3.5.
3. The remaining ultrapure water was added to the solution to dilute to the gauge level.

### Comparative Example 4

An amount of AAV in the purified elution fraction (Liquid B) was determined in the same manner as in Comparative Example 1. The result is presented in FIG. 4 (left) and FIG. 5 (left).

### Comparative Example 5

An amount of AAV in the purified elution fraction (Liquid B) was determined in the same manner as in Example 2, except that as Liquid B, a 10 mM acetic acid solution of pH 3.5 was used instead of the 10 mM glycine/10 mM sodium acetate solution of pH 4.5. The result is presented in FIG. 4 (middle).

### Production Method of 10 mM Acetic Acid Solution of pH 3.5

1. Acetic acid was weighed out in a beaker to be 10 mM, and was dissolved in approximately 80% of a total amount of ultrapure water to be used.
2. Hydrochloric acid was added to the above solution to adjust to pH 3.5.
3. The remaining ultrapure water was added to the solution to dilute to the gauge level.
   Purification Evaluation of AAV8 with Amino Acid-Containing Solution and Infectivity Evaluation of Purified AAV8

### Example 7

An amount of AAV in the purified elution fraction (Liquid B) was determined in the same manner as in Example 2. The result is presented in FIG. 5 (right).

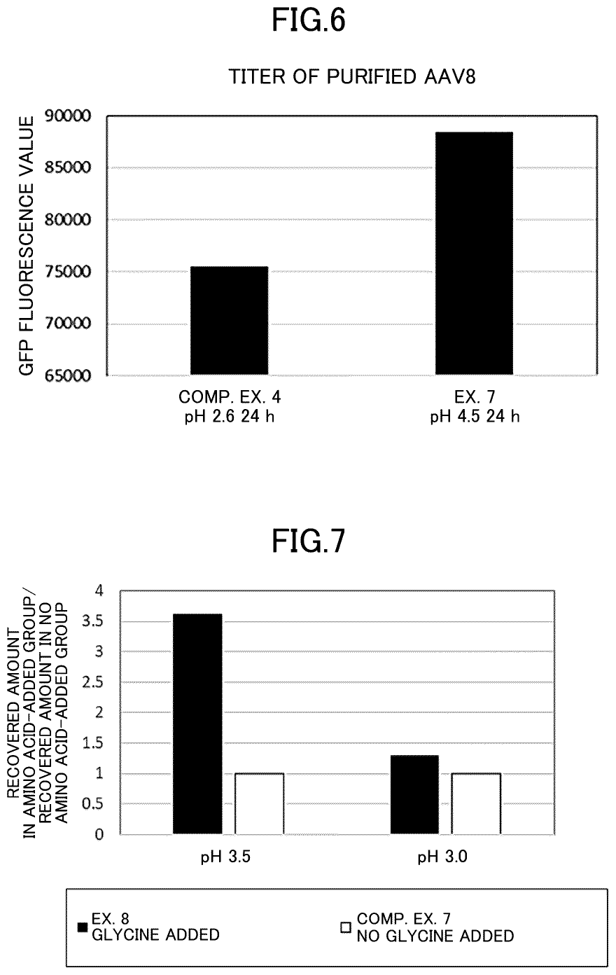

Twenty four hours after obtaining the eluent, the eluent was neutralized. HEK293T cells were infected with the eluent, and the titer was measured 72 hours later. The result is presented in FIG. 6 (right).

### Comparative Example 6

An amount of AAV in the purified elution fraction (Liquid B) was determined in the same manner as in Comparative Example 2. The result is presented in FIG. 5 (center).

Twenty four hours after obtaining the eluent obtained in Comparative Example 4, the eluent was neutralized. HEK293T cells were infected with the neutralized eluent, and the titer was measured 72 hours later. The result is presented in FIG. 6 (left).

It was found from the results of FIGS. 4 and 5 that, when elution was performed with the amino acid-containing solution of pH 3 or higher and pH 7 or lower (Examples 6 and 7), AAV8 could be efficiently recovered from the elution fraction compared to the case where no amino acid was added (Comparative Examples 5 and 6), and the efficiency thereof was equal to or better than the case where the solution of lower than pH 3 was used (Comparative Example 4).

It was found from the results of FIG. 6 that, when the elution was performed with the amino acid-containing solution of pH 3 or higher and pH 7 or lower (Example 7), the higher titer was retained than that in the case where the elution was performed with the solution of lower than pH 3 (Comparative Example 4).

Purification Evaluation of AAV with Amino Acid-Containing Solution of pH 3.0 to pH 6.0

### Example 8

Whether AAV2 could be eluted from a column with an amino acid-containing solution was evaluated using the purified AAV2 liquid prepared in Production Example 7.

Tricorn (registered trademark) 5/50 (manufactured by Cytiva) was packed with Capto (registered trademark) AVB (manufactured by Cytiva) to thereby prepare a column for affinity exchange purification.

Liquids A to G below were prepared, and were each passed through a 0.2 μm filter before use.

The purified AAV2 was loaded onto Capto (registered trademark) AVB (manufactured by Cytiva), and an amount of AAV detected from the elution fraction was evaluated.
Liquid A: 1×TD buffer (137 mM sodium chloride, 8.1 mM disodium hydrogen phosphate, 2.68 mM potassium chloride, and 1.47 mM potassium dihydrogen phosphate)-0.2% Poloxamer 188 solution (manufactured by Sigma-Aldrich)
Liquid B: 10 mM glycine/10 mM acetic acid solution of pH 3.5
Liquid C: 10 mM glycine/10 mM acetic acid solution of pH 3.0
Liquid D: 0.1 M glycine-hydrochloric acid solution of pH 2.6
Liquid E: 0.01 M sodium hydroxide solution
Liquid F: PAB solution (120 mM phosphoric acid, 167 mM acetic acid, and 2.2 mM benzyl alcohol)
Liquid G: clarified AAV2 liquid

The 0.1 M glycine-hydrochloric acid solution of pH 2.6 was produced by the method described in Production Example 4, and the 10 mM glycine/10 mM acetic acid solution of pH 3.5 was produced by the method described in Example 6.

### Production Method of 10 mM Glycine/10 mM Acetic Acid Solution of pH 3.0

1. A 10 mM glycine/10 mM acetic acid solution was produced.
2. Hydrochloric acid was added to the above solution to adjust to pH 3.0.

The above column was connected to AKTA (registered trademark) Avant 25 (manufactured by Cytiva), and was equilibrated with Liquid A. Thereafter, Liquid G was passed through to allow the column carrier to retain AAV thereon, followed by washing with Liquid A, and AAV was eluted with Liquid B, and Liquid C in this order. The column was washed with Liquid D, Liquid E, and Liquid F.

The AAV of the purified elution fractions (Liquid B and Liquid C) were each neutralized with a 1 M Tris buffer, followed by determining an amount of AAV 2 in the liquid through quantitative PCR (qPCR) using QuantStudio3 real-time PCR system (manufactured by Thermo Fisher Scientific Inc.) in the same manner as in Production Example 4. For the determination of the AAV2 amount, AAVpro Titration Kit (for Real Time PCR) Ver. 2 (registered trademark) was used. The result is presented in FIG. 7 (black bars).

### Comparative Example 7

Amounts of AAV of the purified elution fractions (Liquid B and Liquid C) were each determined in the same manner as in Example 8, except that as Liquid B, a 10 mM acetic acid solution of pH 3.5 was used instead of the 10 mM glycine/10 mM acetic acid solution of pH 3.5, and as Liquid C, a 10 mM acetic acid solution of pH 3.0 was used instead of the 10 mM glycine/10 mM acetic acid solution of pH 3.0. The results are presented in FIG. 7 (white bars).

The 10 mM acetic acid solution of pH 3.5 was produced by the method described in Comparative Example 5.

### Production Method of 10 mM Acetic Acid Solution of pH 3.0

1. A 10 mM acetic acid solution was produced.
2. Hydrochloric acid was added to the above solution to adjust to pH 3.0.

It was found from the results of FIG. 7 that, when the elution was performed with the amino acid-containing solution of pH 3.0 to pH 3.5 (Example 8), the recovery rate of the purified AAV2 from the elution fraction was increased compared to the case where no amino acid was added (Comparative Example 7).

### Example 9

Whether AAV2 could be eluted from a column with an amino acid-containing solution was evaluated using the clarified AAV2 liquid prepared in Production Example 7.

Tricorn (registered trademark) 5/20 (manufactured by Cytiva) was packed with Capto (registered trademark) AVB (manufactured by Cytiva) to thereby prepare a column for affinity exchange purification.

Liquids A to F below were prepared, and were each passed through a 0.2 μm filter before use.

The purified AAV2 was loaded onto the Capto (registered trademark) AVB (manufactured by Cytiva), and an amount of AAV detected from the elution fraction was evaluated.
Liquid A: 1×TD buffer (137 mM sodium chloride, 8.1 mM disodium hydrogen phosphate, 2.68 mM potassium chloride, and 1.47 mM potassium dihydrogen phosphate)-0.2% Poloxamer 188 solution (manufactured by Sigma-Aldrich)
Liquid B: 10 mM glycine/10 mM malic acid solution of pH 4.0
Liquid C: 0.1 M glycine-hydrochloric acid solution of pH 2.6
Liquid D: 0.01 M sodium hydroxide solution
Liquid E: PAB solution (120 mM phosphoric acid, 167 mM acetic acid, and 2.2 mM benzyl alcohol)
Liquid F: clarified AAV2 liquid

The 0.1 M glycine-hydrochloric acid solution of pH 2.6 was produced by the method described in Production Example 4.

### Production Method of 10 mM Glycine/10 mM Malic Acid Solution of pH 4.0

1. A 10 mM glycine/10 mM malic acid solution was produced.
2. Hydrochloric acid was mixed with the above solution to adjust to pH 4.0.

The above column was connected to AKTA (registered trademark) Avant 25 (manufactured by Cytiva), and equilibrated with Liquid A. Thereafter, Liquid F was passed through to allow the column carrier to retain AAV thereon, followed by washing with Liquid A, and AAV was eluted with Liquid B. The column was washed with Liquid C, Liquid D, and Liquid E.

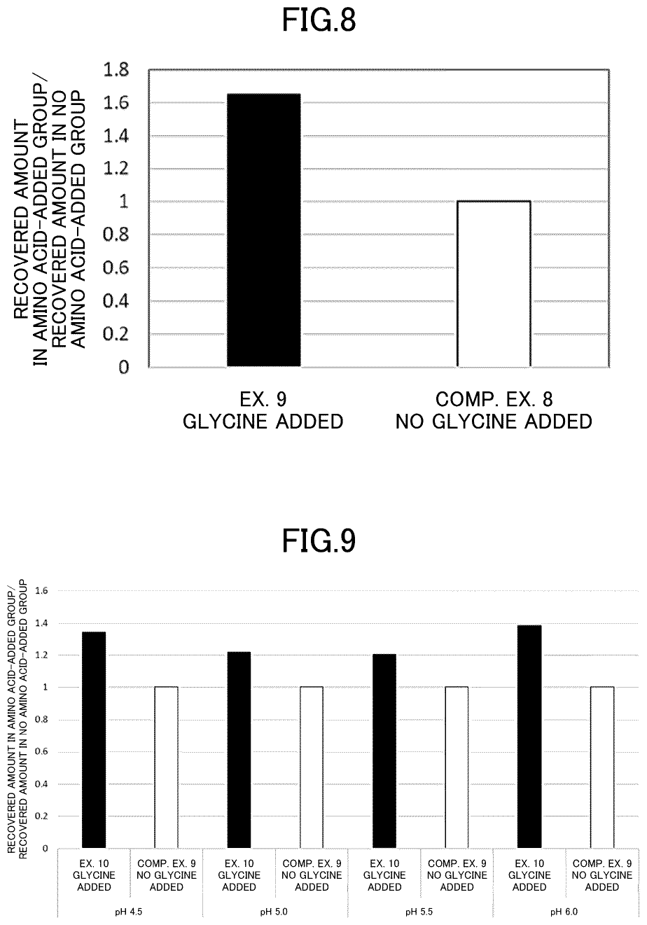

The AAV of the purified elution fraction (Liquid B) was neutralized in a 1 M Tris buffer, followed by determining an amount of AAV8 in the liquid through quantitative PCR (qPCR) using QuantStudio3 real-time PCR system (manufactured by Thermo Fisher Scientific Inc.) in the same manner as in Production Example 4. For the determination of the AAV amount, AAVpro Titration Kit (for Real Time PCR) Ver. 2 (registered trademark) was used. The result is presented in FIG. 8 (black bar).

### Comparative Example 8

An amount of AAV in the purified elution fraction (Liquid B) was determined in the same manner as in Example 9, except that as Liquid B, a 10 mM malic acid solution of pH 4.0 was used instead of the 10 mM glycine/10 mM malic acid solution of pH 4.0. The result is presented in FIG. 8 (white bar).

### Production Method of 10 mM Malic Acid Solution of pH 4.0

1. A 10 mM malic acid solution was produced.
2. Sodium hydroxide was added to the above solution to adjust to pH 4.0.

It was found from the results of FIG. 8 that, when the elution was performed with the amino acid-containing solution of pH 4.0 (Example 9), the recovery rate of the purified AAV2 from the elution fraction was increased compared to the case where no amino acid was added (Comparative Example 8).

### Example 10

An amount of AAV in the purified elution fraction (Liquid B) was determined in the same manner as in Example 9, except that as Liquid B, a 10 mM glycine/10 mM malic acid solution of pH 4.5, a 10 mM glycine/10 mM malic acid solution of pH 5.0, a 10 mM glycine/10 mM malic acid solution of pH 5.5, or a 10 mM glycine/10 mM malic acid solution of pH 6.0 was used instead of the 10 mM glycine/10 mM malic acid solution of pH 4.0. The results are presented in FIG. 9 (black bars).

### Production Method of 10 mM Glycine/10 mM Malic Acid Solution of pH 4.5, 10 mM Glycine/10 mM Malic Acid Solution of pH 5.0, 10 mM Glycine/10 mM Malic Acid Solution of pH 5.5, or 10 mM Glycine/10 mM Malic Acid Solution of pH 6.0

1. A 10 mM glycine/10 mM malic acid solution was produced.
2. Sodium hydroxide was added to the above solution to adjust to pH 4.5, pH 5.0, pH 5.5, or pH 6.0.

### Comparative Example 9

An amount of AAV in the purified elution fraction (Liquid B) was determined in the same manner as in Example 10, except that as Liquid B, a 10 mM malic acid solution of pH 4.5, a 10 mM malic acid solution of pH 5.0, a 10 mM malic acid solution of pH 5.5, or a 10 mM malic acid solution of pH 6.0 was used instead of the 10 mM glycine/10 mM malic acid solution of pH 4.5, the 10 mM glycine/10 mM malic acid solution of pH 5.0, the 10 mM glycine/10 mM malic acid solution of pH 5.5, or the 10 mM glycine/10 mM malic acid solution of pH 6.0. The results are presented in FIG. 9 (white bars).

### Production Method of 10 mM Malic Acid Solution of pH 4.5, 10 mM Malic Acid Solution of pH 5.0, 10 mM Malic Acid Solution of pH 5.5, or 10 mM Malic Acid Solution of pH 6.0

1. A 10 mM malic acid solution was produced.
2. Sodium hydroxide was added to the above solution to adjust pH 4.5, pH 5.0, pH 5.5, or pH.

It was found from the results of FIG. 9 that, when the elution was performed with the amino acid-containing solution of pH 4.5 to pH 6.0 (Example 10), the recovery rate of the purified AAV2 from the elution fraction was increased compared to the case where no amino acid was added (Comparative Example 9).

Purification Evolution of AAV8 with Amino Acid-Containing Solution of pH 6.5 to pH 7.0

### Example 11

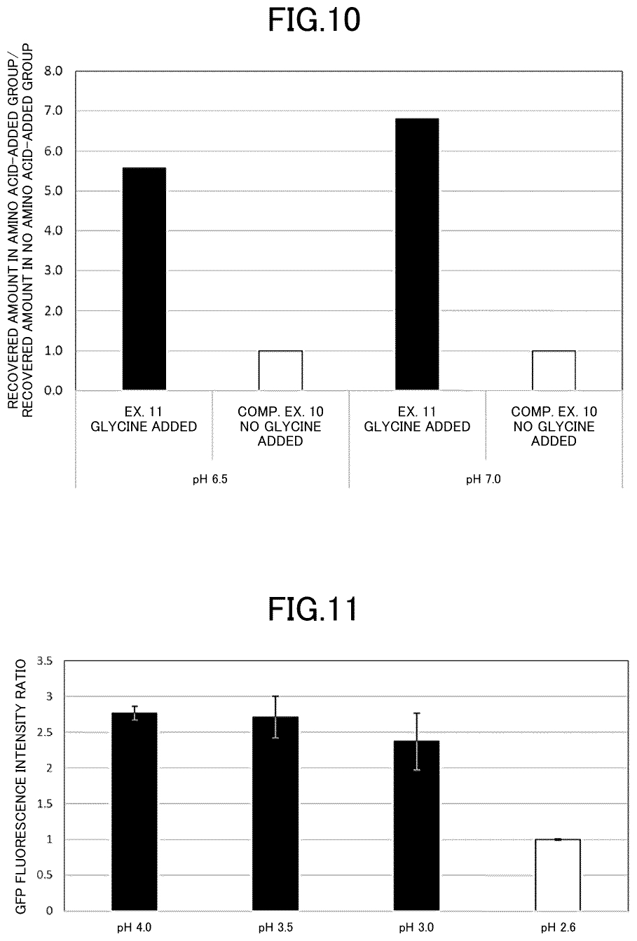

An amount of AAV in the purified elution fraction (Liquid B) was determined in the same manner as in Example 2, except that as Liquid B, a 10 mM glycine/10 mM sodium phosphate solution of pH 6.5 or a 10 mM glycine/10 mM sodium phosphate solution of pH 7.0 was used instead of the 10 mM glycine/10 mM sodium acetate solution of pH 4.5. The results are presented in FIG. 10 (black bars).

### Production Method of 10 mM Glycine/10 mM Sodium Phosphate Solution of pH 6.5 or 10 mM Glycine/10 mM Sodium Phosphate Solution of pH 7.0

1. A 10 mM glycine/10 mM sodium dihydrogenphosphate dihydrate solution and a 10 mM glycine/10 mM disodium hydrogen phosphate dodecahydrate were produced.
2. The above two solutions were mixed to produce a 10 mM glycine/10 mM sodium phosphate solution of pH 6.5 or a 10 mM glycine/10 mM sodium phosphate solution of pH 7.0.

### Comparative Example 10

An amount of AAV in the purified elution fraction (Liquid B) was determined in the same manner as in Example 11, except that as Liquid B, a 10 mM sodium phosphate solution of pH 6.5 or a 10 mM sodium phosphate solution of pH 7.0 was used instead of the 10 mM glycine/10 mM sodium phosphate solution of pH 6.5 or the 10 mM glycine/10 mM sodium phosphate solution of pH 7.0. The results are presented in FIG. 10 (white bars).

### Production Method of 10 mM Sodium Phosphate Solution of pH 6.5 or 10 mM Sodium Phosphate Solution of pH 7.0

1. A 10 mM phosphoric acid solution and a 10 mM sodium phosphate solution were produced.
2. The above two solutions were mixed to produce a 10 mM sodium phosphate solution of pH 6.0 or 10 mM sodium phosphate solution of pH 7.0.

It was found from the results of FIG. 10 that, when the elution was performed with the amino acid-containing solution of pH 6.5 to pH 7.0 (Example 11), the recovery rate of the purified AAV8 from the elution fraction was increased compared to the case where no amino acid was added (Comparative Example 10).

Infectivity Evaluation of AAV2 Purified with Amino Acid-Containing Solution

### Example 12

A purified elution fraction (Liquid B) was obtained in the same manner as in Example 10, except that as Liquid B, a 10 mM glycine/10 mM malic acid solution of pH 3.0, a 10 mM glycine/10 mM malic acid solution of pH 3.50, or 0, a 10 mM glycine/10 mM malic acid solution of pH 4.0 was used instead of the 10 mM glycine/10 mM malic acid solution of pH 4.5, the 10 mM glycine/10 mM malic acid solution of pH 5.0, the 10 mM glycine/10 mM malic acid solution of pH 5.5, or the 10 mM glycine/10 mM malic acid solution of pH 6.0.

Twenty four hours later, the obtained eluent was neutralized. HEK293T cells were infected with the neutralized eluent, and a titer was measured 72 hours later. The results are presented in FIG. 11 (black bars) (standard errors are indicated with error bars).

### Production Method of 10 mM Glycine/10 mM Malic Acid Solution of pH 3.0, 10 mM Glycine/10 mM Malic Acid Solution of pH 3.5, or 10 mM Glycine/10 mM Malic Acid Solution of pH 4.0

1. A 10 mM glycine/10 mM malic acid solution was produced.
2. Hydrochloric acid was added to adjust to pH 3.0, pH 3.5, or pH 4.0.

### Comparative Example 11

A purified elution fraction (Liquid B) was obtained in the same manner as in Example 10, except that as Liquid B, a 10 mM glycine-hydrochloric acid solution of pH 2.6 was used instead of the 10 mM glycine/10 mM malic acid solution of pH 4.5, the 10 mM glycine/10 mM malic acid solution of pH 5.0, the 10 mM glycine/10 mM malic acid solution of pH 5.5, or the 10 mM glycine/10 mM malic acid solution of pH 6.0.

Twenty four hours later, the obtained eluent was neutralized. HEK293T cells were infected with the neutralized eluent, and a titer was measured 72 hours later. The result is presented in FIG. 11 (white bar) (a standard error is indicated with an error bar).

### Production Method of 10 mM Glycine-Hydrochloric Acid Solution of pH 2.6

1. Glycine was weighed out in a beaker to be 10 mM, and was dissolved in approximately 80% of a total amount of ultrapure water to be used.
2. Hydrochloric acid was added to the above solution to adjust to pH 2.6.

It was found from the results of FIG. 11 that, when the elution was performed with the amino acid-containing solution of pH 3 or higher and pH 4 or lower (Example 12), the high titer was retained compared to the case where elution was performed with the solution of lower than pH 3 (Comparative Example 11). Although it is not presented as data, it can be easily assumed that a high titer can be retained when a solution of pH higher than pH 4 is used, compared to a case where elution is performed with a solution of lower than pH 3.

Purification Evaluation of AAV2 with Amino Acid-Containing Solution

### Example 13

Whether AAV2 could be eluted from a bead carrier with an amino acid-containing solution was evaluated using the clarified AAV2 liquid prepared in Production Example 7.

Capto (registered trademark) AVB (manufactured by Cytiva) was dispensed into Eppendorf tubes.

Liquids A to D below were prepared, and were each passed through a 0.2 μm filter before use.

The purified AAV2 was loaded onto Capto (registered trademark) AVB (manufactured by Cytiva) in the Eppendorf tube, and an amount of AAV detected from the elution fraction was evaluated.
Liquid A: 1×TD buffer (137 mM sodium chloride, 8.1 mM disodium hydrogen phosphate, 2.68 mM potassium chloride, and 1.47 mM potassium dihydrogen phosphate)-0.2% Poloxamer 188 solution (manufactured by Sigma-Aldrich)
Liquid B: 10 mM glycine/10 mM acetic acid solution of pH 3.5
Liquid C: 0.1 M glycine-hydrochloric acid solution of pH 2.6
Liquid D: clarified AAV2 liquid

The 0.1 M glycine-hydrochloric acid solution of pH 2.6 was produced by the method described in Production Example 4, and the 10 mM glycine/10 mM acetic acid solution of pH 3.5 was produced by the method described in Example 6.

Liquid A was added to the Eppendorf tube into which the carrier was dispensed to equilibrate. Then, centrifuge was performed and the resultant supernatant was removed. Thereafter. Liquid D was added, and the resultant mixture was loaded onto the carrier while stirring by a rotator at 4° C. for 16 hours. After allowing the carrier to retain AAV thereon, the carrier was washed with Liquid A three time, and AAV was eluted with Liquid B. Thereafter, the carrier was washed with Liquid C.

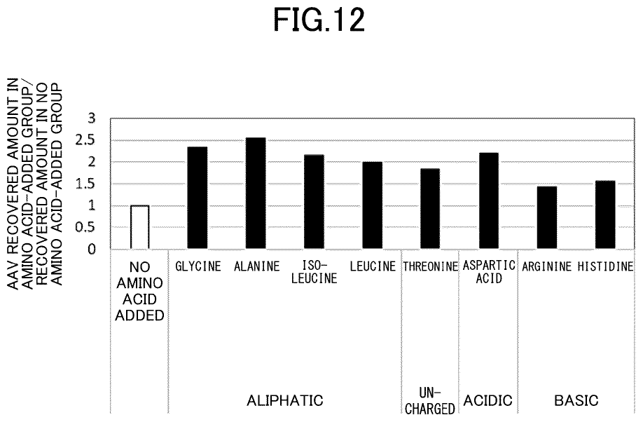

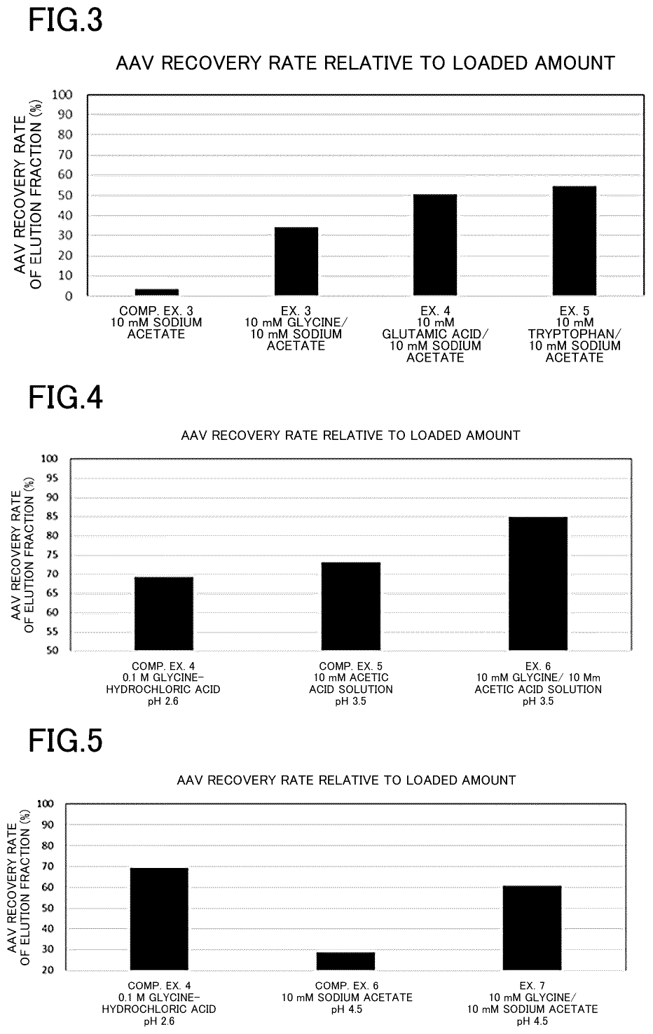

An amount of AAV2 in the purified elution fraction (Liquid B) was determined in the liquid through quantitative PCR (qPCR) using QuantStudio3 real-time PCR system (manufactured by Thermo Fisher Scientific Inc.) in the same manner as in Production Example 4. The determination of the AAV2 amount was performed using AAVpro Titration Kit (for Real Time PCR) Ver. 2 (registered trademark). The result is presented in FIG. 12 (black bar, the first from the left).

### Example 14

An amount of AAV in the purified elution fraction (Liquid B) was determined in the same manner as in Example 13, except that as Liquid B, a 10 mM alanine/10 mM acetic acid solution of pH 3.5 was used instead of the 10 mM glycine/10 mM acetic acid solution of pH 3.5. The result is presented in FIG. 12 (black bar, the second from the left).

### Production Method of 10 mM Alanine/10 mM Acetic Acid Solution of pH 3.5

1. A 10 mM alanine/10 mM acetic acid solution was produced.
2. Hydrochloric acid was added to the above solution to adjust to pH 3.5.

### Example 15

An amount of AAV in the purified elution fraction (Liquid B) was determined in the same manner as in Example 13, except that as Liquid B, a 10 mM isoleucine/10 mM acetic acid solution of pH 3.5 was used instead of the 10 mM glycine/10 mM acetic acid solution of pH 3.5. The result is presented in FIG. 12 (black bar, the third from the left).

### Production Method of 10 mM Isoleucine/10 mM Acetic Acid Solution of pH 3.5

1. A 10 mM isoleucine/10 mM acetic acid solution was produced.
2. Hydrochloric acid was added to the above solution to adjust to pH 3.5.

### Example 16

An amount of AAV in the purified elution fraction (Liquid B) was determined in the same manner as in Example 13, except that as Liquid B, a 10 mM leucine/10 mM acetic acid solution of pH 3.5 was used instead of the 10 mM glycine/10 mM acetic acid solution of pH 3.5. The result is presented in FIG. 12 (black bar, the fourth from the left).

### Production Method of 10 mM Leucine/10 mM Acetic Acid Solution OF PH 3.5

1. A 10 mM leucine/10 mM acetic acid solution was produced.
2. Hydrochloric acid was added to the above solution to adjust to pH 3.5.

### Example 17

An amount of AAV in the purified elution fraction (Liquid B) was determined in the same manner as in Example 13, except that as Liquid B, a 10 mM threonine/10 mM acetic acid solution of pH 3.5 was used instead of the 10 mM glycine/10 mM acetic acid solution of pH 3.5. The result is presented in FIG. 12 (black bar, the fourth from the right).

### Production Method of 10 mM Threonine/10 mM Acetic Acid Solution of pH 3.5

1. A 10 mM threonine/10 mM acetic acid solution was produced.
2. Hydrochloric acid was added to the above solution to adjust to pH 3.5.

### Example 18

An amount of AAV in the purified elution fraction (Liquid B) was determined in the same manner as in Example 13, except that as Liquid B, a 3 mM aspartic acid/10 mM acetic acid solution of pH 3.5 was used instead of the 10 mM glycine/10 mM acetic acid solution of pH 3.5. The result is presented in FIG. 12 (black bar, the third from the right).

### Production Method of 3 mM Aspartic Acid/10 mM Acetic Acid Solution of pH 3.5

1. A 3 mM aspartic acid/10 mM acetic acid solution was produced.
2. Hydrochloric acid was added to the above solution to adjust to pH 3.5.

### Example 19

An amount of AAV in the purified elution fraction (Liquid B) was determined in the same manner as in Example 13, except that as Liquid B, a 10 mM arginine/10 mM acetic acid solution of pH 3.5 was used instead of the 10 mM glycine/10 mM acetic acid solution of pH 3.5. The result is presented in FIG. 12 (black bar, the second from the right).

### Production Method of 10 mM Arginine/10 mM Acetic Acid Solution of pH 3.5

1. A 10 mM arginine/10 mM acetic acid solution was produced.
2. Hydrochloric acid was added to the above solution to adjust to pH 3.5.

### Example 20

An amount of AAV in the purified elution fraction (Liquid B) was determined in the same manner as in Example 13, except that as Liquid B, a 10 mM histidine/10 mM acetic acid solution of pH 3.5 was used instead of the 10 mM glycine/10 mM acetic acid solution of pH 3.5. The result is presented in FIG. 12 (black bar, the first from the right).

### Production Method of 10 mM Histidine/10 mM Acetic Acid Solution of pH 3.5

1. A 10 mM histidine/10 mM acetic acid solution was produced.
2. Hydrochloric acid was added to the above solution to adjust to pH 3.5.

### Comparative Example 12

An amount of AAV in the purified elution fraction (Liquid B) was determined in the same manner as in Example 13, except that as Liquid B, a 10 mM acetic acid solution of pH 3.5 was used instead of the 10 mM glycine/10 mM acetic acid solution of pH 3.5. The result is presented in FIG. 12 (white bar).

The 10 mM acetic acid solution of pH 3.5 was produced by the method described in Comparative Example 5.

It was found from the results of FIG. 12 that, when the elution was performed with the solution including various aliphatic amino acids, uncharged amino acids, acidic amino acids, or basic amino acids, other than glycine (Examples 13 to 20), AAV2 could be efficiently recovered from the elution fraction compared to the case where no amino acid was added (Comparative Example 12).

For example, embodiments of the present invention include the following.

<1> A method of producing an adeno-associated virus (AAV), the method including:
separating an adeno-associated virus (AAV), which is bonded to an affinity carrier, from the affinity carrier using an amino acid-containing solution of pH 3 or higher and pH 7 or lower.

<2> The method of producing the adeno-associated virus (AAV) according to <1>,
wherein an amino acid included in the amino acid-containing solution is an aliphatic amino acid, an en-i-sei amino acid, an acidic amino acid, an aromatic amino acid, or an uncharged amino acid.

<3> The method of producing the adeno-associated virus (AAV) according to <2>,
wherein the amino acid is the aliphatic amino acid, the acidic amino acid, or the aromatic amino acid.

<4> The method of producing the adeno-associated virus (AAV) according to any one of <1> to <3>,
wherein the amino acid-containing solution of pH 3 or higher and pH 7 or lower further includes a pH adjuster.

<5> A method of purifying an adeno-associated virus (AAV), the method including:
separating an adeno-associated virus (AAV), which is bonded to an affinity carrier, from the affinity carrier using an amino acid-containing solution of pH 3 or higher and pH 7 or lower.

<6> A method of inhibiting a reduction in an infectious titer of an adeno-associated virus (AAV), the method including:
separating an adeno-associated virus (AAV), which is bonded to an affinity carrier, from the affinity carrier using an amino acid-containing solution of pH 3 or higher and pH 7 or lower.

<7> An eluent for separating an adeno-associated virus (AAV) bonded to an affinity carrier from the affinity carrier, the eluent including:
an amino acid-containing solution of pH 3 or higher and pH 7 or lower.

This international application claims priority based on Japanese Patent Application No. 2022-054065 filed on Mar. 29, 2022, the entire contents of which are incorporated herein by reference.

1. A method of producing an adeno-associated virus (AAV), the method comprising:
separating an adeno-associated virus (AAV), which is bonded to an affinity carrier, from the affinity carrier using an amino acid-containing solution of pH 3 or higher and pH 7 or lower.

2. The method of producing the adeno-associated virus (AAV) according to claim 1,
wherein an amino acid included in the amino acid containing solution is an aliphatic amino acid, a basic amino acid, an acidic amino acid, an aromatic amino acid, or an uncharged amino acid.

3. The method of producing the adeno-associated virus (AAV) according to claim 2,
wherein the amino acid is the aliphatic amino acid, the acidic amino acid, or the aromatic amino acid.

4. The method of producing the adeno-associated virus (AAV) according to claim 1,
wherein the amino acid-containing solution of pH 3 or higher and pH 7 or lower further includes a pH adjuster.

5. A method of purifying an adeno-associated virus (AAV), the method comprising:
separating an adeno-associated virus (AAV), which is bonded to an affinity carrier, from the affinity carrier using an amino acid-containing solution of pH 3 or higher and pH 7 or lower.

6. A method of inhibiting a reduction in an adeno-associated virus (AAV) infectious titer, the method comprising:
separating an adeno-associated virus (AAV), which is bonded to an affinity carrier, from the affinity carrier using an amino acid-containing solution of pH 3 or higher and pH 7 or lower.

7. (canceled)

8. The method of producing the adeno-associated virus (AAV) according to claim 1, further comprising:
binding the adeno-associated virus (AAV) with an antibody that is included in the affinity carrier,
wherein a pH of the amino acid-containing solution of pH 3 or higher and pH 7 or lower is pH 3.5 or higher and pH 5.5 or lower.

9. The method of purifying the adeno-associated virus (AAV) according to claim 5, further comprising:
binding the adeno-associated virus (AAV) with an antibody that is included in the affinity carrier,
wherein a pH of the amino acid-containing solution of pH 3 or higher and pH 7 or lower is pH 3.5 or higher and pH 5.5 or lower.

10. The method of inhibiting the reduction in the infectious titer of the adeno-associated virus (AAV) according to claim 6, further comprising:
binding the adeno-associated virus (AAV) with an antibody that is included in the affinity carrier,
wherein a pH of the amino acid-containing solution of pH 3 or higher and pH 7 or lower is pH 3.5 or higher and pH 5.5 or lower.
